(19) United States
(12) Patent Application Publication
(10) Pub. No.: US 2008/0299127 A1
(43) Pub. Date: Dec. 4, 2008

(54) PROTECTIVE STAPHYLOCOCCUS AUREUS VACCINE BASED ON CELL WALL-ASSOCIATED PROTEINS

(76) Inventors: Martin Kroenke, Cologne (DE); Oleg Krut, Huerth (DE); Eva Glowalla, Cologne (DE); Bettina Tosetti, Cologne (DE)

Correspondence Address: Ballard Spahr Andrews & Ingersoll, LLP, SUITE 1000, 999 PEACHTREE STREET, ATLANTA, GA, 30309-3915, US

(21) Appl. No.: 12/115,282

(22) Filed: May 5, 2008

(30) Foreign Application Priority Data
May 4, 2007 (EP) 07107512.1

(51) Int. Cl. A61K 39/395; A61K 39/00; C07K 7/00; C12N 15/11; C12N 5/06; A61P 37/00; C12P 21/04; C12N 15/00; C07K 16/18; G01N 33/569

(52) U.S. Cl. 424/139.1; 424/243.1; 424/190.1; 530/300; 530/387.9; 435/7.33; 536/23.7; 435/320.1; 435/325; 435/69.3

(57) ABSTRACT

The present invention relates to a pharmaceutical composition or a medicament, notably a protective Staphylococcus aureus vaccine, comprising at least one cell wall-associated Staphylococcus aureus protein or a fragment or derivative thereof causing an immune response that induces opsonophagocytic activity of human neutrophils for S. aureus. The invention further provides particular cell wall-associated Staphylococcus aureus proteins and their use.

### CROSS REFERENCE TO RELATED APPLICATIONS

This application claims priority to European Patent application No. EP 07107512.1, filed May 4, 2007, which application is hereby incorporated by this reference in its entirety.

### FIELD OF THE INVENTION

The present invention relates to a pharmaceutical composition or a medicament, notably a protective Staphylococcus aureus vaccine, comprising at least one cell wall-associated Staphylococcus aureus protein or a fragment or derivative thereof causing an immune response that induces opsonophagocytic activity of human neutrophils for S. aureus. The invention further provides particular cell wall-associated Staphylococcus aureus proteins and their use.

### BACKGROUND OF THE INVENTION

Staphylococcus aureus is a nosocomial as well as a community-acquired pathogen, which causes several diseases, ranging from minor skin infections to serious life-threatening wound infections, bacteraemia, endocarditis, pneumonia and toxic shock syndrome (Lowy F. D., N. Engl. J. Med., 339(8):520-32 (1998)). The worldwide growing incidence of staphylococcal infections is strongly related to the increased use of surgical devices and a growing number of immunocompromised patients. The situation has become more serious, since the increased use of antibiotics led to the emergence of methicillin-resistant S. aureus strains (MRSA) (Selvey L. A. et al., Infect. Control. Hosp. Epidemiol., 21(10):645-8 (2000); Peacock J. E. et al., Ann. Intern. Med., 93(4):526-32 (1980)). More recently S. aureus isolates with reduced susceptibility to vancomycin (VISA), the antibiotic of choice against MRSA strains, were described (Tenover F. C. et al., Emerg. Infect. Dis., 7(2):327-32 (2001); Tenover F. C. et al., J. Clin. Microbiol., 36(4):1020-7 (1998)), followed by first reports about vancomycin-resistant clinical isolates (VRSA) in 2002 (Staphylococcus aureus resistant to vancomycin—United States, 2002. MMWR Morb Mortal Wkly Rep 2002; 51(26):565-7; Palazzo I. C. et al., J. Clin. Microbiol., 43(1):179-85 (2005)). The rising emergence of multidrug-resistant staphylococci led to a growing interest in the development of alternative approaches to prevent and treat staphylococcal infections.

The human humoral (Stranger-Jones Y. K. et al., Proc. Natl. Acad. Sci., USA, 103(45):16942-7 (2006)) immune response to staphylococcal infection is a crucial component of the host defense (Bredius R. G. et al., J. Immunol., 151(3):1463-72 (1993); Liese J. G. et al., Lancet, 347(8996):220-3 (1996)). Recent investigations on antibody responses to S. aureus in patients during the acute phase of infections as well as in healthy individuals showed a high titer of opsonising antibodies against the pathogen in both groups (Dryla A. et al., Clin. Diagn. Lab. Immunol., 12(3):387-98 (2005); Royan S. et al., FEMS Immunol. Med. Microbiol., 29(4):315-21 (2000)). However, antibodies against certain staphylococcal antigens were missing or underrepresented in patient serum, whereas high levels of these antibodies were present in healthy donors (Dryla A. et al., Clin. Diagn. Lab. Immunol., 12(3):387-98 (2005); Clarke S. R. et al., J. Infect. Dis., 193(8):1098-108 (2006)). The humoral immune response elicited during staphylococcal infection does not provide efficient protection against subsequent infections. Likewise, vaccination with whole cells of killed S. aureus did not protect animals and humans against staphylococcal infection (Greenberg D. P. et al., Infect. Immun., 55(12):3030-4 (1987); Poole-Warren L. A. et al., Clin. Nephrol., 35(5):198-206 (1991)). Therefore the interest in the design of effective vaccines against S. aureus is growing.

Vaccination with capsular polysaccharides (CP) demonstrated reduced severity of staphylococcal infections in animals and humans (Fattom A. et al., Vaccine, 23(5):656-63 (2004); Fattom A. I. et al., Infect. Immun., 64(5):1659-65 (1996)). Since vaccines based on capsular polysaccharides induce only serotype-specific protection, a bivalent vaccine against S. aureus serotype 5 and 8 was developed, which comprises about 75% of the most infectious strains. Due to the poor immunogenicity of polysaccharides, conjugation to a carrier protein was required to induce humoral immune responses. Tested in clinical trials, the CP vaccine elicited high antibody levels in dialysis patients, though not leading to a significant long term protection against bacteraemia (Fattom A. I. et al., Vaccine, 22(7):880-7 (2004)). Alternatively, immunisation with known staphylococcal virulence factors provided a protective immune response against S. aureus in animal models. A specific humoral response raised against single extracellular matrix binding proteins like clumping factor A (ClfA), fibronectin-binding protein (FNBP) or collagen-binding protein (Cna) conferred a reduced susceptibility to staphylococcal colonisation (Mamo W. et al., Microbiol. Immunol., 44(5):381-4 (2000); Mamo W. et al., Vaccine, 12(11):988-92 (1994)). Notwithstanding the positive results achieved, vaccination with a single antigen does not suffice to induce full protection against S. aureus infection. Additionally, a high variation in the expression pattern of virulence factors among S. aureus strains hampers the development of an universal vaccine.

Recently is has been shown that a multivalent vaccine consisting of four antigenic determinants provides protection against lethal challenge with S. aureus (Stranger-Jones Y. K. et al., Proc. Natl. Acad. Sci., USA, 103(45):16942-7 (2006)). These data call for the identification of highly antigenic surface proteins conserved among S. aureus strains, which could be potential targets for a polyvalent vaccine. Indeed, numerous staphylococcal surface proteins, predicted to be promising antigenic targets, have been identified so far using recently adopted technologies, like proteomics (Brady R. A. et al., Infect. Immun., 74(6):3415-26 (2006); Gatlin C. L. et al., Proteomics, 6(5):1530-49 (2006); Pieper R. et al., Proteomics, 6(15):4246-58 (2006); Vytvytska O. et al., Proteomics, 2(5):580-90 (2002); Nandakumar R. et al., J. Proteome Res., 4(2):250-7 (2005)) or protein selection methods based on expression libraries (Clarke S. R. et al., J. Infect. Dis., 193(8):1098-108 (2006); Etz H. et al., Proc. Natl. Acad. Sci. USA, 99(10):6573-8 (2002); Weichhart T. et al., Infect. Immun., 71(8):4633-41 (2003); Yang G. et al., Vaccine, 24(8):1117-23 (2006)). Unfortunately, most studies lack functional proof in vivo. By serological proteome analysis Vytvytska et al. identified several highly immunogenic proteins using individual sera from patients and healthy donors (Vytvytska O. et al., Proteomics, 2(5):580-90 (2002)).

IVIG preparations are successfully used in the treatment of patients with antibody deficiencies, in order to reduce their high susceptibility to bacterial infections (Lamari F. et al., J. Pharm. Biomed. Anal., 22(6):1029-36 (2000)). Since such preparations consist of purified IgGs derived from not less than thousand plasma donors, they are characterised by a broad spectrum of opsonising antibodies against various pathogens including S. aureus. The administration of antibodies against bacterial surface antigens provides opsonisation, complement activation and the phagocytosis of the bacteria (Lamari F. et al., J. Pharm. Biomed. Anal., 22(6):1029-36 (2000); Ono Y. et al., J. Infect. Chemother., 10(4):234-8 (2004)). With regard to S. aureus infections, polyclonal IVIG preparations selected for high titers of anti-ClfA IgGs (SA-IGIV) or of anti-ClfA in combination with anti-SdrG IgGs (INH-A21), enhanced bacterial clearance in experimental animals, when used in combination with vancomycin treatment or alone. (Vernachio J. et al., Antimicrob. Agents Chemother., 47(11):3400-6 (2003); Vernachio J. H. et al., Antimicrob. Agents Chemother., 50(2):511-8 (2006)). Even though preparations were selected for high levels of one specific antibody type, they are polyclonal and thus it is unlikely that the protective activity is caused by the interaction of a single type of specific antibodies.

### SUMMARY OF THE INVENTION

The world-wide growing incidence of multidrug-resistant S. aureus encourages the research for effective vaccination strategies. Since immunisation with vaccines based on single, known components of the staphylococcal virulence apparatus or staphylococcal carbohydrates achieved only partial benefits in prevention of S. aureus infections (Mamo W. et al., Microbiol. Immunol., 44(5):381-4 (2000); Mamo W. et al., Vaccine, 12(11):988-92 (1994); Brouillette E. et al., 20(17-18):2348-57 (2002); Zhou H. et al., Vaccine, 24(22):4830-7 (2006)), the identification of novel antigenic proteins remains an important issue.

To overcome the limitation of antibody source intravenous immunoglobulin preparations (IVIG) were used that contain a broad spectrum of opsonising antibodies against various pathogens including S. aureus. Employing 2-DE gel electrophoresis, subtractive immunoblotting and mass spectrometry several cell wall-associated proteins were identified as novel potential vaccine protein targets. This method for the identification of potential vaccines was coined SUPRA (subtractive proteome analysis). The protective activity of antibodies raised against some of the identified proteins was demonstrated in vitro and in a murine sepsis model. The invention thus provides

(1) a pharmaceutical composition or a medicament comprising at least one cell wall-associated Staphylococcus aureus protein or a fragment or derivative thereof causing an immune response that induces opsonophagocytic activity of human neutrophils for S. aureus;
(2) in a preferred embodiment of (1) above, the cell wall-associated Staphylococcus aureus protein is selected from SEQ ID NOs: 1-39 and fragments and derivatives thereof;
(3) in a preferred embodiment of (1) or (2) above the pharmaceutical composition or medicament is a protective Staphylococcus aureus vaccine;
(4) the use of the cell wall-associated S. aureus protein or a fragment or derivative thereof as defined in (1) or (2) above for preparing a protective Staphylococcus aureus vaccine;
(5) a specific cell wall-associated Staphylococcus aureus protein selected from SEQ ID NOs: 1, 3-5, 7, 9, 11-13, 16-18, 20, 22, 28-32, 36-39 or a fragment or derivative thereof;
(6) an antibody against a protein as defined in (5) above;
(7) a diagnostic reagent, a pharmaceutical composition or a medicament comprising at least one antibody as defined in (6) above or an antibody against the protein defined in (2) above;
(8) a DNA sequence encoding the cell wall-associated S. aureus protein or a fragment or derivative thereof as defined in (5) above;
(9) a vector carrying the DNA as defined in (8) above;
(10) a host cell transformed with the vector as defined in (9) above and/or carrying the DNA as defined in (8) above;
(11) a method for producing the cell wall-associated S. aureus protein or a fragment or derivative thereof as defined in claim (5) above, which method comprises culturing the host cell as defined in (10) above;
(12) the use of an antibody as defined in (6) above or an antibody against the protein defined in (2) above for preparing a medicament for treating Staphylococcus aureus infection in a human or animal;
(13) a method for vaccinating a human or animal against Staphylococcus aureus which comprises administering the human or animal with cell wall-associated S. aureus protein or a fragment or derivative thereof as defined in (1) above; and.
(14) a method for treating Staphylococcus aureus infection in a human or animal, which comprises administering the human or animal at least one antibody as defined in (6) above or an antibody against the protein defined in (2) above.

### FIGURES

FIG. 1: In vitro opsonophagocytosis of GFP-expressing S. aureus by human PMNs. Human PMNs, bacteria (MOI 10) and IVIG, dSaIVIG and heat-inactivated serum (HIserum), respectively, were incubated at 37° C. for 3 minutes. Flow cytometric analysis revealed the percentage of GFP-positive human PMNs. Dead human PMNs were stained by propidium iodide (PI).

FIG. 2: Differences in the S. aureus antigen recognition profiles using IVIG and IVIG depleted of S. aureus-specific IgGs (dSaIVIG) for immunoblotting. Cell wall-associated proteins were isolated from S. aureus strain ATCC 29213. Proteins were separated according to their isoelectric point (pI) on pI 3-10 IPG strips (A) and on pI 4-7 IPG strips (B). Gels loaded with 100 μg protein were used for subtractive immunoblotting. Spots not immunoreactive with dSaIVIG (top), but with IVIG (middle) represented spots predicted to be promising vaccine candidates (circles). Spots immunoreactive with both, IVIG and dSaIVIG were excluded from further investigation (squares). For MALDI-TOF identification corresponding spots were matched with preparative gels loaded with 500 μg protein (bottom). Circled and numbered spots identified by MALDI-TOF are listed in Table 1.

FIG. 3: SDS PAGE of purified recombinant Enolase (rEno), Oxo (rOxo) and hp2160 (rhp2160) stained with silver (A) and specificity control of affinity purified anti-Eno, anti-Oxo and anti-hp2160 antibodies by immunoblotting (B). Cytplasmic protein fraction of E. coli expressing recombinant rEno, rOxo or rhp2160 (1), affinity purified rEno, rOxo or rhp2160 protein (2), and cytoplasmic protein fraction of non-transformed E. coli (3) were separated by SDS PAGE. 10 μg of cytoplasmic fractions and 2.5 μg of purified proteins were loaded on the gel. Immunoblots were probed with affinity purified anti-Eno, anti-Oxo or anti-hp2160 (1 mg/ml).

FIG. 4: In vitro opsonising activity of purified anti-Eno, -Oxo and -2160 IgGs in comparison to IVIG and heat-inactivated serum (HIserum). Human PMNs, bacteria (MOI 10) and 2.5% HIserum or 500 μg/ml IgGs were incubated for 3 min at 37° C. for opsonophagocytosis. Human PMNs and bacteria without opsonins served as negative control (dark grey). Phagocytosis was stopped by differential centrifugation and the cells were applied to flow cytometric analysis to determine the green fluorescence at t3 (light grey). Incubation was continued at 37° C. for further 30 min (t33, transparent), followed by flow cytometric analysis of the human PMNs. The percentage of GFP-positive human PMNs represents cells with internalised GFP-expressing S. aureus (M1).

FIG. 5: In vitro opsonophagocytic killing of S. aureus opsonised with anti-Eno IgGs (open squares), anti-Oxo IgGs (triangles) or IVIG (circles). Human PMNs, bacteria (MOI 1) and 2.5% IgGs (500 μg/ml) were incubated at 37° C. for 3 min. Cells were lysed immediately after differential centrifugation (t3) as well as 30 min (t33) and 60 min (t63) later. The mean and SD of triplicates are represented.

FIG. 6: Detection of antibody levels in serum of mice immunised with rEno (open circles), rOxo (triangles), rhp2160 (diamonds) and BSA (squares) as mock-control using ELISA. 10-fold serial dilutions of sera from each mouse were applied to maxisorb plates coated with 5 μg/ml antigen. As background (circles) signal neg. serum of non-immunised mice was used. Results are expressed as OD at 450 nm. Represented is the mean and SD for a group of 7 mouse sera.

FIG. 7: In vivo imaging of S. aureus Xen29 infection in C57BL/6 mice immunised with rEno, rOxo, rhp2160 and BSA as mock-control. S. aureus Xen 29 (1×10⁸ CFU) was injected i.v. The immunised mice (n=7) were shaved, to avoid the loss of photon emission and imaged ventrally. (A). The process of infection was monitored daily by 5-min exposure over a 3-day course by using the IVIS system. (B). Quantitative analysis of the bioluminescence determined as a calculation of a total number of photons/sec within a ROI using the IVIS computer software (untreated, circles; BSA, squares; Eno, open circles; Oxo, triangles; hp2160, diamonds). Represented is mean and SD of a group of seven mice. (C) Bacterial load in organs of immunised mice 3 days after S. aureus challenge. Each bar represents mean and SD for a group of 7 mice (BSA, black; Eno, hatched; Oxo, transparent; hp2160, light grey). Asterisks indicate a statistically significant difference compared to BSA controls.

FIG. 8: Bacteriostatic effect of IVIG on S. aureus growth. IVIG absorbed with S. aureus (dSaIVIG, light grey triangle) did not influence S. aureus growth, whereas IVIG absorbed with E. coli (dEcIVIG, dark grey circle) still displayed a bacteriostatic effect.

FIG. 9: Bacteriostatic effect of affinity purified anti-Eno (dark grey square), anti-Oxo (dark grey triangle) and anti-hp2160 (dark grey diamond) antibodies on S. aureus growth.

### DETAILED DESCRIPTION OF THE INVENTION

As high anti-staphylococcal antibody levels are present in both healthy individuals and acutely infected patients, but with heterogeneous distribution pattern (Dryla A. et al., Clin. Diagn. Lab. Immunol., 12(3):387-98 (2005)), it was assumed that commercially available intravenous immunoglobulin preparation (IVIG) might contain an antibody repertoire of choice for the identification of potential vaccine candidates. Thus, an intravenous immunoglobulin (IVIG) preparation was employed as a source of antibodies directed against S. aureus surface proteins for the identification of novel vaccine protein candidates. IVIG induced a strong opsonophagocytic activity of human neutrophils for S. aureus. In order to identify proteins that are targeted by IVIG, subtractive proteome analysis (SUPRA) of S. aureus surface proteins was performed. Proteins solely reacting with IVIG, but not with IVIG depleted of S. aureus-specific opsonising antibodies (dSaIVIG), were identified as vaccine candidates using MALDI-TOF analysis. Three out of 40 identified candidates, enolase (Eno), oxoacyl reductase (Oxo) and hypothetical protein (hp2160), were expressed as GST-fusion proteins, purified and used for the enrichment of corresponding IgGs from IVIG by affinity chromatography. Affinity purified anti-Eno, anti-Oxo and anti hp2160 antibodies showed significant opsonising activity enabling uptake and killing of S. aureus by human neutrophils. Significant antibody responses were elicited in mice immunised with recombinant antigens. After challenge with S. aureus, reduced staphylococcal spread was detected in immunised mice by in vivo imaging system. The recovery of S. aureus CFUs from organs of immunised mice was diminished by 10-100-fold. The results of this study suggest our approach to be a valuable tool for the identification of novel vaccine candidates and therapeutic antibodies. Thus, each of these S. aureus proteins proved to be able to induce an efficient immune response, establishing protective immunity to S. aureus.

In the pharmaceutical composition or medicament as defined in (1) to (3) above the cell wall-associated protein is preferably derived from S. aureus strain ATCC 29213 (American type culture collection) (commercially available at ATCC, Manassas, Va. (USA)). Furthermore it is preferred that the cell wall associated protein solely reacts with an intravenous immunoglobulin (iVIG) preparation but not with an iVIG preparation depleted of S. aureus-specific opsonising antibodies.

It is particularly preferred that the cell wall-associated protein is selected from the esterase-like protein (hp 2160) of SEQ ID NO: 1, the enolase (Eno) of SEQ ID NO: 2, the 3-oxoacyl-reductase (Oxo) of SEQ ID NO: 3, the NADH dehydrogenase like protein of SEQ ID NO: 4, the anion-binding protein like protein of SEQ ID NO: 5, the peptidoglycan hydrolase of SEQ ID NO: 6, the conserved hypothetical protein of SEQ ID NO: 7, the immunodominant antigen A of SEQ ID NO: 8, the aldehyde dehydrogenase of SEQ ID NO: 9, the truncated beta-hemolysin of SEQ ID NO: 10, the secretory antigen precursor SsaA homolog of SEQ ID NO: 11, the 50S ribosomal protein L13 of SEQ ID NO: 12, the conserved hypothetical protein of SEQ ID NO: 13, the translational elongation factor TU of SEQ ID NO: 14, the autolysin of SEQ ID NO: 15, the hypothetical protein of SEQ ID NO: 16, the protoporphyrinogen oxidase of SEQ ID NO: 17, synergohymenotropic toxin precursor-like protein of SEQ ID NO: 18, the glutamyl-t-RNAGln amidotransferase subunit A of SEQ ID NO: 19, the aminotransferase NifS homologue of SEQ ID NO: 20, the translational elongation factor TU of SEQ ID NO: 21, the hypothetical protein of SEQ ID NO: 22, the immunodominant antigen A of SEQ ID NO: 23, the ATP synthase beta chain of SEQ ID NO: 24, the alanine dehydrogenase of SEQ ID NO: 25, the phosphopentomutase of SEQ ID NO: 26, the NAD-specific glutamate dehydrogenase of SEQ ID NO: 27, autolysin precursor-like protein of SEQ ID NO: 28, the chorismate mutase homolog of SEQ ID NO: 29, the SceD precursor-like protein of SEQ ID NO: 30, the hypothetical protein of SEQ ID NO: 31, the uridylate kinase of SEQ ID NO: 32, the transcription pleiotropic repressor codY of SEQ ID NO: 33, the enterotoxin SEM of SEQ ID NO: 34, the ferrichrome transport ATP-binding protein of SEQ ID NO: 35, the conserved hypothetical protein of SEQ ID NO: 36, the 50S ribosomal protein L25 of SEQ ID NO: 37, the triosephosphate isomerase of SEQ ID NO: 38, the Xaa-Pro dipeptidase of SEQ ID NO: 39 and fragments and derivatives thereof. Particularly preferred among those proteins is the Eno of Seq ID NO: 2, the Oxo of Seq ID NO: 3, hp2160 of Seq ID NO: 1 or a fragment or derivative thereof.

The term “fragments” within the meaning of the invention comprises a continuous segment out of the above proteins having at least 5 amino acid residues, preferably at least 10 amino acid residues.

The term “derivative” within the meaning of the invention comprises deletion, substitution and addition muteins of the above proteins and combinations thereof. A deletion mutein lacks up to 10, preferably up to 5 continuous or separate amino acid residues, relative to the starting protein sequence. A substitution mutein has substituted up to 10, preferably up to 5 continuous or separate amino acid residues, relative to the starting protein sequence. An addition mutein has added up to 10, preferably up to 5 continuous or separate amino acid residues, relative to the starting protein sequence. The term “derivative” also covers chemical modifications of the protein (e.g. the modification of functional groups, linking of functional groups (such as alkylation, hydroxylation, phosphatation, thiolation, carboxilation and the like), linkage to at least one further functional protein domain (such as marker proteins, carrier proteins, proteins holding adjuvant properties and the like; the linkage being directly or via a linker molecule) and to other biologically active molecules (toxins, antibiotics, lipids, carbohydrates, nucleic acids and the like).

All of the modifications do, however, not reduce the immunogenic activity of the protein. The pharmaceutical composition or medicament as defined in (1) to (3), (4), (7) and (12) above may further comprise pharmaceutically acceptable carriers (as well as pharmaceutically acceptable binders, stabilizers, excipients, etc.) which are required for the administration of the pharmaceutical composition or medicament to the human or animal patients.

The proteome analysis revealed nearly 40 cell wall-associated vaccine candidates, which were specifically recognised by opsonising IgGs. A number of the identified proteins are already known as staphylococcal virulence factors, like the fibrinogen-binding, bone sialoprotein-binding protein (Yacoub A. et al., Eur. J. Biochem., 222(3):919-25 (1994)), β-hemolysin (Hedstrom S. A. et al., Acta. Pathol. Microbiol. Immunol. Scand. [B], 90(3):217-20 (1982)) or enterotoxin M (SEM) (Jarraud S. et al., J. Immunol., 166(1):669-77 (2001)). Other proteins, like staphylococcal enolase or alanine dehydrogenase, are known as cytosolic proteins, but are also located on the surface of S. aureus or other bacterial pathogens (Caneiro C. R. et al., Microbes. Infect., 6(6):604-8 (2004); Rosenkrands I. et al., J. Bacteriol., 184(13):3485-91 (2002)). IsaA and EF-Tu are proteins, already identified by serological proteome analysis (SERPA) (Vytvytska O. et al., Proteomics, 2(5):580-90 (2002)). It is important to note that our selection of S. aureus potential vaccine candidates is distinct from 15 S. aureus proteins previously identified by Vytvytska et al. employing SERPA (Vytvytska O. et al., Proteomics, 2(5):580-90 (2002)). Unfortunately, these authors provided little information about the potential immunochemical or protective properties of these candidates.

On the other hand the proteins according to embodiment (5) of the invention were not known in the art.

In the study which lead to the invention enolase (Eno), oxoacyl-reductase (Oxo) and the hypothetical protein (hp2160) were repeatedly identified by 2-DE immunoblots. Affinity purified antibodies against each of these proteins were shown to be able to opsonise bacteria, leading to a potent opsonophagocytosis and killing effect in vitro. Strong humoral immune response was elicited in mice immunised with the individual proteins. The high level of specific antibodies, detected in mice sera, correlated with the reduction of bacterial infection in vaccinated animals. With regard to these results, the combination of several S. aureus antigens is expected to produce even better and more robust immunity.

Lately, Enolase was shown to be located on the cell surface of S. aureus and to bind extracellular matrix components like laminin (Carneiro C. R. et al., Microbes. Infect., 6(6):604-8 (2004)) and plasminogen (Molkanen T. et al., FEBS Lett., 517(1-3):72-8 (2002)); its important role as staphylococcal virulence factor seems indisputable. Immunisation with recombinant enolase successfully reduced staphylococcal colonisation of S. aureus. The protective activity of enolase seems to be not limited to S. aureus. Recently it was demonstrated that mice immunised with enolase were protected against S. epidermidis colonisation (Sellman B. R. et al., Infect. Immun., 73(10):6591-600 (2005)). The two other proteins, Oxo and hp2160 are not yet described. Based on sequence homologies, Oxo displays characteristics of enzymes, involved in fatty acid biosynthesis and the hp2160 sequence shows similarity to esterase, an enzyme with lipolytic activity. It is noteworthy that enolase and other S. aureus vaccine candidates have been missed by a recent elegant search for protective vaccine candidates by Stranger-Jones et al. and co-workers employing a bioinformatics-based reverse vaccinology approach (Stranger-Jones Y. K. et al., Proc. Natl. Acad. Sci., USA, 103(45):16942-7 (2006)). These authors screened multiple staphylococcal genomes for genes containing the conserved LPXTG motif that is common for surface proteins covalently linked to the cell wall. The assumption was that many proteins involved in S. aureus pathogenesis are anchored to the bacterial cell wall by a transpeptidation mechanism that requires the C-terminal sorting signal with a conserved LPXTG motif (Navarre W. W. et al., Microbiol. Mol. Biol. Rev., 63(1):174-229 (1999)). Indeed, mice immunised with a combination of four such antigens, previously tested for their distinct immunogenic properties, were protected against lethal challenge with clinical S. aureus isolates. However, numerous novel, anchorless proteins with adhesive and invasive properties that do not contain either a signal peptide or the LPXTG motif were identified in the past few years. The mechanism, by which these proteins are secreted and reassociated with the surface is still unknown (Chhatwal G. S. et al., Trends Microbiol., 10(5):205-8 (2002)). Next to Enolase, a protein exhibiting GAPDH activity and called Tpn was identified on the staphylococcal surface. Tpn was demonstrated to possess transferrin-binding activity (Modun B. et al., Infect. Immun., 67(3):1086-92 (1999)). Another anchorless, broad-spectrum adhesin Eap (Hansen U. et al., Matrix Biol., 25(4):252-60 (2006); Palma M. et al., J. Bacteriol., 181(9):2840-5 (1999)) binds to a large variety of host components, recognising not only fibronectin, fibrinogen or prothrombin, but also collagens, laminin and other extracellular matrix proteins (Palma M. et al., J. Bacteriol., 181(9):2840-5 (1999); McGavin M. H. et al., Infect. Immun., 61(6):2479-85 (1993)). Each of these anchorless proteins was shown to play an important role in the pathogenesis of S. aureus, which underscores the continued necessity to search for vaccine candidates lacking the LPXTG motif.

According to embodiment (6) the invention also provides antibodies against the protein of (5) above (said antibodies may be polyclonal or monoclonal). Such antibodies, as well as the antibodies against the proteins of (2) above (which are employed in the diagnostic reagent, pharmaceutical composition or medicament of (7) above or the medicament of (12) above or the method of (14) above) can be prepared (i.e. raised against the antigen) by suitable methods known to a skilled person.

In the diagnostic reagent of embodiment (7) the antibody may be linked to a suitable detection moiety (such as dye or enzyme), allowing the protein to be deleted. Alternatively, it may also be linked to suitable carrier.

The DNA sequence of (8) above preferably comprise the nucleotide sequence of SEQ ID NOs:45 to 84 above.

The host cell in (10) above may be any prokaryotic or eukaryotic cell, prokaryotic cells such as E. coli being, however, preferred.

The method of embodiment (11) furthermore comprises collecting the desired protein from the host (in case it is not secreted) or the culture broth.

The invention will be further explained in the following Examples, which are, however, not to be construed to limit the invention.

### EXAMPLES

Bacterial strains: S. aureus strain ATCC 29213 was obtained from the American Type Culture Collection (ATCC) and was used for the extraction of cell wall-associated proteins. For opsonophagocytosis assays GFP-expressing S. aureus (ATCC 29213), generated in our laboratory (Schnaith A. et al., J. Biol. Chem. 282(4):2695-706 (2007)), was used. The bioluminescent S. aureus Xen29, applied for in vivo imaging studies, was purchased from Xenogen Corp. (Alameda, USA). This strain was derived from the virulent S. aureus ATCC 12600 strain and is characterised by its possibility to synthesise the luciferase enzyme as well as its substrate constitutively (Francis K. P. et al., Infect. Immun., 68(6):3594-600 (2000); Kadurugamuwa J. L. et al., Infect. Immun., 71(2):882-90 (2003)). For cloning and expression of recombinant proteins Escherichia coli strains Top 10, DH5α and BL21 (Invitrogen Corp., Karlsruhe, Germany) were used.

Isolation of cell wall-associated proteins: S. aureus ATCC 29213 from agar plate was precultured in Luria-Bertani (LB) broth at 37° C. with constant shaking to log growth. An aliquot of the preculture was inoculated into 200 ml of LB broth to obtain a starting optical density (OD₆₀₀) of 0.05. The bacteria were cultured with constant shaking at 37° C. under aerobic conditions until early exponential growth phase was reached. Bacteria were pelleted by centrifugation at 17,000×g for 20 min at 4° C. and the pellets washed twice in 10 mM TE-buffer, pH 8.0. The cell wall-associated proteins were extracted according to the method of Antelmann (Antelmann H. et al., Proteomics, 2(5):591-602 (2002)) with modifications. The cell pellets were resuspended in 10 ml 1.5 M LiCl, 25 mM Tris-HCl, pH 7.2 containing protease inhibitors (Roche Diagnostics, Mannheim, Germany) and incubated on ice for 30 min. After centrifugation at 17,000×g for 20 min at 4° C., supernatants were pooled and precipitated with 10% w/v trichloroacetic acid (Sigma, Munich, Germany) overnight (ON) at 4° C. The precipitate was collected by centrifugation at 17,000×g for 60 min, and 4° C., washed three times with ice-cold ethanol and dried under vacuum. Extracted proteins were dissolved in 8M urea for two-dimensional gel electrophoresis (2-DE). The protein concentration was determined using the Bio-Rad DC protein assay kit (Bio-Rad, Germany) according to the instructions of the manufacturer.

SDS-PAGE: One-dimensional gel electrophoresis (1-DE) was performed with 10% polyacrylamide gels according to the method of Schägger and Jagow (Schagger H. et al., Anal. Biochem., 166(2):368-79 (1987)) using the Criterion Cell (Bio-Rad). The gels were stained either with Coomassie brilliant blue R250 (CBB) dissolved in 45% ethanol and 15% acetic acid or with silver according to the method described by Shevchenko et al. (Shevchenko A. et al., Anal. Chem., 68(5):850-8 (1996)). Western blots were performed using the Criterion Blotter (Bio-Rad) according to the instruction of the manufacturer.

Depletion of S. aureus specific IgGs from IVIG: Human intravenous polyclonal immunoglobulin G (IVIG) was purchased from Octapharma (Octagam®, Langenfeld, Germany). To deplete S. aureus/E. coli specific antibodies from IVIG (dSaIVIG and dEcIVIG, respectively), S. aureus (ATCC 29213) or E. coli (DH5α) from overnight culture were pelleted by centrifugation at 17,000×g for 20 min, 4° C., washed twice in 1× PBS and the pellet was resuspended in IVIG. The suspension was rotated slowly overnight at 4° C. Bacteria were removed by centrifugation and the supernatant was passed through a 0.2 μm membrane filter, followed by the determination of the protein concentration.

Subtractive proteome analysis (SUPRA): 2-DE was performed according to the method described by Bernardo et al. (Bernardo K. et al., Antimicrob. Agents Chemother., 48(2):546-444 (2004)) using the Multiphor II (Pharmacia-FRG) system according to the instructions of the manufacturer. Proteins dissolved in 8 M urea were separated on 18 cm immobilised pH gradient (IPG) strips in a nonlinear pH range of 3-10 and 4-7 (GE Healthcare, Munich, Germany). Isoelectric focusing (IEF) was performed using 500 μg protein for coomassie and 100 μg for silver gels and western blots. Proteins were solubilised in rehydration buffer (8 M urea, 2 M thiourea, 40 mM DTT, 1% CHAPS, 0.5% Pharmalyte and separated according to their pIs (isoelectric point) using the Multiphor II. After IEF, rod gels were equilibrated first in equilibration buffer (50 mM Tris-HCL [pH 8.8], 8 M urea, 2 M thiourea, 30% glycerol, 2% SDS, 0.05% bromophenol blue) containing 3.5 mg/ml DTT, followed by equilibration in equilibration buffer containing 45 mg/ml iodoacetamide and applied to SDS-PAGE. The proteins were separated on 12.5% Tris-glycine SDS gels (25 cm, 20 cm, 1.0 mm) using the Ettan Dalt II system (GE Healthcare).

Immunoblotting: Proteins separated by 2-DE were transferred to nitrocellulose membranes using the Trans-Blot Cell (Bio-Rad) according to the instruction of the manufacturer. After blocking in 5% low-fat dry milk and 2% BSA dissolved in TBST (Tris-buffered saline-Tween 20, pH 7.5) for 1 h at room temperature, membranes were probed with IVIG (1:500) or with IVIG depleted of S. aureus specific IgGs (dSaIVIG) using an equal antibody concentration, overnight at 4° C. Membranes were washed in TBST and incubated with anti-human IgG peroxidase conjugate (1:2500) for specific detection of immune complexes. After subsequent washing in TBST, signals were developed using the ECL detection system (GE Healthcare). Each membrane was used twice. After treatment with dSaIVIG, membrane was stripped at 50° C. using stripping buffer containing 62.5 mM Tris-HCl pH 6,8; 2% SDS; 100 mM β-Mercaptoethanol, and incubated with IVIG.

Signals of interest were matched with corresponding spots on the CBB stained preparative gel, excised and digested with trypsin (Bernardo K. et al., Antimicrob. Agents Chemother., 48(2):546-444 (2004)). MALDI-TOF/MS analysis was performed using the Bruker REFLEX IV mass spectrometer (Bruker-Daltonik, Leipzig, Germany). Further data processing was done using the XMASS 5.1.1 postanalysis software. Protein mass spectra were analysed using the MASCOT software against a public database (NCBI)

Cloning of GST-fusion proteins: The recombinant proteins, Enolase (rEno; SEQ ID NO: 2), Oxoacyl-Reductase (rOxo; SEQ ID NO: 3) and the hypothetical protein hp2160 (rhp2160; SEQ ID NO: 1) were cloned as N-terminal GST-fusion proteins using the Gatetway Technology (Invitrogen) according to the instructions of the manufacturer. The open reading frame (ORF) of each gene was amplified from S. aureus (ATCC 29213) chromosomal DNA by PCR using 5′-CACCATGCCAATTATTA CAGATG-3′ (SEQ ID NO: 40) and 5′-TTATTTATCT AAGTTATAGAA TGATTTG-3′(SEQ ID NO: 41), 5′-CACCATGAAA ATGACTAAGA GTGCT-3′ (SEQ ID NO: 42) and 5′-TACATGTACA TTCCACCATT TACATG-3′ (SEQ ID NO: 43), and 5′-CACCTTGATT AGAAACCGTG TTATG-3′ (SEQ ID NO: 44) and 5′-TTAGTTATTT TGTGTTACAT CCTCATC-3′ (SEQ ID NO: 45) for Eno, Oxo and hp2160, respectively. The reaction products were cloned into pENTR/D-TOPO and transformed into E. coli TOP10 (Invitrogen). By LR recombination the genes of interest were transferred from entry clone into pDEST15 destination vector, in order to generate a vector coding for the N-terminal GST-fusion protein. After transformation of the destination vector into E. coli DH5α, positive clones were identified by restriction analysis and transformed into the E. coli BL21 strain for expression.

Expression and purification of staphylococcal antigens: E. coli BL21 harbouring the recombinant plasmids were grown in LB medium containing ampicillin at 30° C. until the cultures reached an OD₆₀₀ of ˜0.6. The protein expression was induced with 0.5 mM IPTG for 4 h at 27° C. Bacterial cells were harvested by centrifugation, washed with 1×PBS and resuspended in 1×PBS containing protease inhibitors and lysed mechanically using the French Press (Thermo Scientific, Waltham, US). The bacterial debris was removed by subsequent centrifugation at 17,000×g. The expression of soluble protein was assessed by SDS-PAGE. The recombinant proteins were purified by affinity chromatography using glutathione sepharose prepacked GSTrap FF columns on the ÄKTApurifier liquid chromatography system (GE Healthcare) according to the manufacturers instructions.

Affinity absorption of specific IgGs from IVIG: In order to enrich specific IgGs, 5-10 mg purified recombinant protein (rEno, rOxo, rhp2160) were covalently linked to the NHS-activated Sepharose according to the manufacturers instructions (GE Healthcare). The absorption of IgGs was performed ON at 4° C. by slow recirculation of IVIG previously transferred into PBS buffer (pH 7.3) using the HiPrep 26/10 Desalting column (GE Healthcare).

Specifically bound IgGs were eluted from the column using 0.1 M Glycine-HCL, pH 2.7 on the ÄKTApurifier liquid chromatography system. The pH of the eluted fractions was neutralised by collecting them into a sufficient amount of 1M Tris-HCL, pH 9. IgG containing fractions were pooled and after buffer exchange into 1×PBS, pH 7.3 concentrated using Centricon Plus-70 centrifugal filter units with a 30.000 MWCO membrane (Millipore, Schwalbach, Germany).

In vitro opsonophagocytosis and bactericidal assay: Human polymorphonuclear cells (PMNs) were isolated by dextran sedimentation and Ficoll-Hypaque gradient centrifugation using 50 ml of heparinised blood from a healthy donor according to standard protocols. Extracted PMNs were resuspended in PBS containing 10 mM glucose, pH7.3, followed by the determination of cell number by trypan blue counting.

For the opsonophagocytosis assay GFP-expressing S. aureus ATCC 29213 from ON culture was inoculated into 25 ml of LB broth and grown at 37° C. to an OD₆₀₀ of 0.3. Bacteria were harvested, washed and adjusted to 1×10⁹ CFU/ml.

IVIG, dSaIVIG or enriched specific IgGs (anti-Eno, -Oxo and -hp2160) were applied in a concentration of 500 μg/ml, which corresponds to the average IgG concentration present in 2.5% human serum.

IgGs, 2.5×10⁶ human PMNs and bacteria in a MOI (multiplicity of infection) of 10 were incubated for 60 s at 37° C. with slow rotation, pelleted by centrifugation for 60 s at 400×g at 37° C. and further incubated for 60 s at 37° C., in order to synchronise phagocytosis. Pellets were resuspended and the phagocytosis was stopped by differential centrifugation for 5 min at 150×g at 4° C. Immediately after centrifugation (t3) an aliquot was removed and stored in a 1:5 dilution in 0.5% BSA/PBS, pH7.3 at 4° C. until FACS analysis was performed. Incubations were continued for further 30 min at 37° C. As a reference value, bacterial uptake in presence of 2.5% heat-inactivated human serum was investigated. Heat-labile complement was destroyed by incubation of the serum for 30 min at 56° C. Triplicates were analysed using the FACSCalibur immunocytometry system and the CELLQuestPro software (BD Biosciences).

To investigate the bactericidal effect induced by IVIG and enriched specific IgGs, 2.5×10⁶ human PMNs and 2.5×10⁶ bacteria (MOI 1) were applied for the killing assay. PMNs, bacteria and IgGs were treated, as described above, until samples immediately (t3) as well as 30 (t33) and 60 minutes after differential centrifugation (t63) were collected. An aliquot of each sample was diluted 1:10 in sterile H₂O and 0.1% Triton X 100 and incubated on ice for 5 min to lyse PMNs. Dilutions of triplicates were spread via spiral plater (Eddy Jet, IUL Instr., Königswinter, Germany) on Miller Hinton (MH) agar plates and the CFU number was determined according to the instructions of the manufacturer. To calculate the percentage of surviving bacteria after 33 and 63 min, CFUs present at t33 or t63 were divided by CFUs present at t3, multiplied by 100.

Immunisation: Female C57/BL6 mice (6-8 weeks) were purchased from Charles River Laboratories (Sulzfeld, Germany). Groups of 7 mice were administered intraperitoneally (i.p.) with an 1:1 emulsion of 100 μg recombinant protein (rEno, rOxo or rhp2160) and complete Freund's Adjuvant (Sigma) in a total volume of 200 μl on day 0, followed by subcutaneous (s.c.) administration of three booster doses using an emulsion of 50 μg antigen and incomplete Freund's Adjuvant (1:1) on day 14, day 28 and day 42. Mice immunised with an emulsion of BSA and adjuvant served as controls. Blood samples were taken from each mouse one week after the last booster injection, in order to determine the antibody titer.

Detection of antibody levels: Enzyme-linked immunoabsorbent assay (ELISA) was used to determine levels of antibodies directed against Eno, Oxo and hp2160 protein in sera of immunised mice. 96-well polystyrene maxisorb plates (Nunc, Wiesbaden, Germany) were coated overnight at 4° C. with 5 mg/ml of the recombinant antigen (rEno, rOxo, rhp2160), BSA or GST in PBS, pH 7.3. The wells were washed three times with PBS containing 0.05% Tween 20 (PBST) and blocked using StartingBlock T20/PBS (Pierce) for 30 min at RT. Triplicates of ten-fold serial dilutions of serum in blocking buffer were incubated for 2 h at RT. After subsequent washing, the bound antibodies were detected with the peroxidase-conjugated goat anti-mouse IgG (Sigma) incubating the samples for 2 h at RT and using the tetramethylbenzidine system (BD Biosciences, Heidelberg, Germany) as substrate according to the manufacturers instructions. The enzyme reaction was stopped by addition of 2NH₂SO₄, followed by measuring the absorbance at OD₄₅₀ using the ELISA plate reader (MRX TC, Dynex Technology, Kaiserslautern, Germany).

Mice sepsis model: The infection of mice was performed using the bioluminescent S. aureus Xen29 strain (Francis K. P. et al., Infect. Immun., 68(6):3594-600 (2000); Kadurugamuwa J. L. et al., Infect. Immun., 71(2):882-90 (2003)). Prior to infection the mice were shaved ventrally to enable light detection. The animals were challenged intravenously (i.v.) with an inoculum of 1×108 CFU S. aureus Xen29 two weeks after the last booster injection. Bacterial inoculum was assessed by plating a serial dilution on MH agar plates and counting the CFU. The infected mice were monitored daily over a time course of 3 days post infection in an anaesthetised state (isofluoran-oxygen gas mixture) using the IVIS imaging system (Xenogen Corp.). Mice were imaged in ventral position for 5 min exposure. An untreated control mouse was included in every image to detect the background photon emission. The infection rate was displayed in a pseudocolour scale representing the number of photons per second emitted from the mice. Highly intensive bioluminescence signals were displayed as red and low intensity signals as blue. The light intensity within defined regions of interest (ROIs) was quantified using the LivingImage software (Xenogen Corp.). Bioluminescent signals were quantified by setting the ROIs over the ventral side of the animal body. Extremities, tail and head were excluded from the analysis.

Bacterial load in organs: To determine the number of bacteria within the organs, mice were sacrificed on day 3 subsequent to the IVIS image. Target tissues (liver, kidney, spleen, heart and lung) were weighed and homogenised in 2 ml PBS containing 0.05% Triton X 100. Ten-fold serial dilutions were spread on MH agar plates and the CFU number was determined.

Statistical analysis: Student's t-tests were performed to analyse the statistical significance of bacterial load in organs. Differences with p<0.05 were considered as statistically significant.

In vitro Bacteriostatic effect: S. aureus ATCC 29213 from ON culture was inoculated into 25 ml of LB broth and grown at 37° C. to an OD₆₀₀ of 0.3. Bacteria were harvested, washed and adjusted to 1×10⁹ CFU/ml. IVIG, dSaIVIG, dEcIVIG or enriched specific IgGs (anti-Eno, -Oxo and -hp2160) were added to LB in a concentration of 200 μg/ml in 500 μl PBS. The prewarmed media were inoculated with 8×103 CFU/ml and the cultures were incubated for 180 min at 37° C. Samples were taken at 0, 60, 90, 120, 150 and 180 minutes post inoculation. Assays without IgGs but LB or PBS or with unspecific murine antiCD8 IgGs served as controls. An aliquot of each sample was diluted 1:2 in 0.1% Triton® X 100 in sterile H₂O and incubated in a sonicator bath for 5 min. Dilutions of triplicates were spread via spiral plater (Eddy Jet, IUL Instr., Königswinter, Germany) on Miller Hinton (MH) agar plates and the CFU number was determined according to the instructions of the manufacturer.

### Results

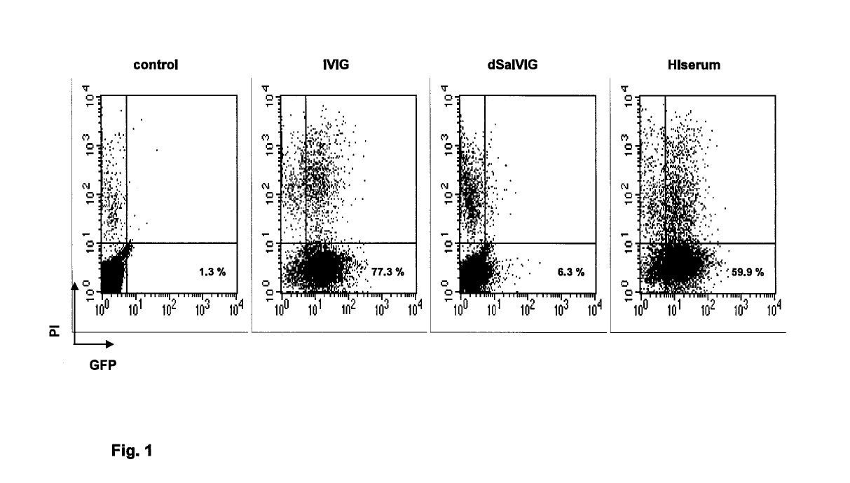

IVIG contains opsonising antibodies to S. aureus: To determine whether commercial IVIG preparations are suitable antibody sources for the identification of immunoreactive antigens, an IVIG preparation (Octagam®) was tested in vitro for opsonising antibodies to S. aureus. The presence of opsonising antibodies was investigated in a PMNs-mediated opsonophagocytosis assay. The efficacy of complement-independent IVIG-mediated phagocytosis was compared to phagocytosis induced by 2.5% heat-inactivated human serum (HIserum). Flow cytometry revealed that IVIG induces strong phagocytic activity of 77.3% human PMNs, which was slightly higher than that mediated by HIserum (59.9%). Phagocytosis was nearly completely abolished, when IVIG was preabsorbed with S. aureus (dSaIVIG) (FIG. 1). These observations provide clear evidence, that opsonising antibodies are abundant in IVIG.

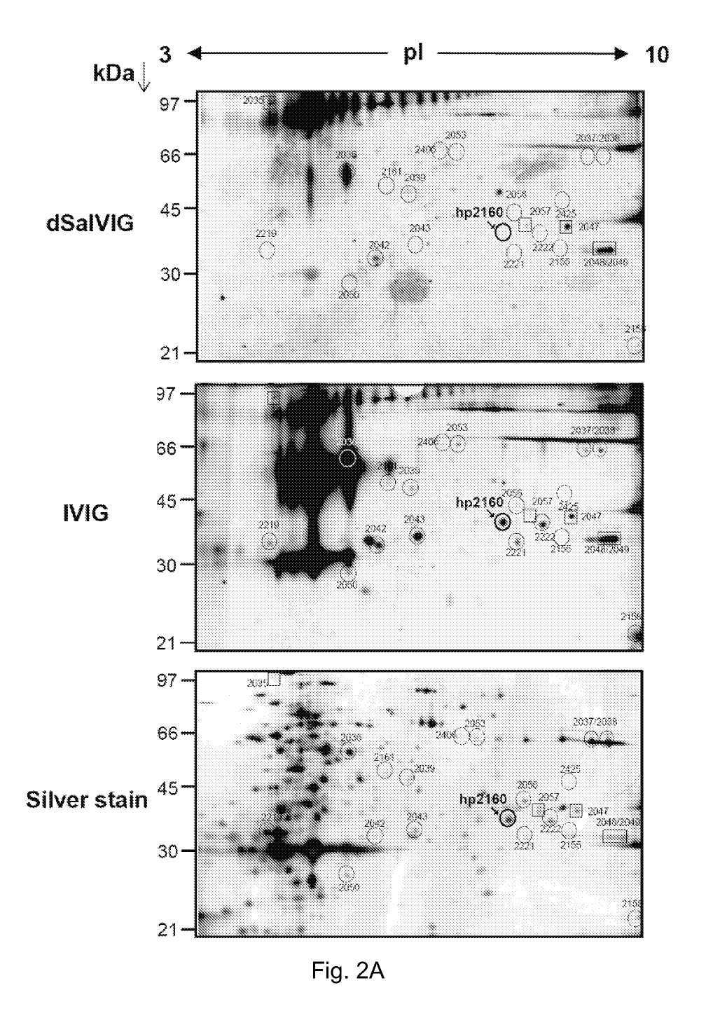

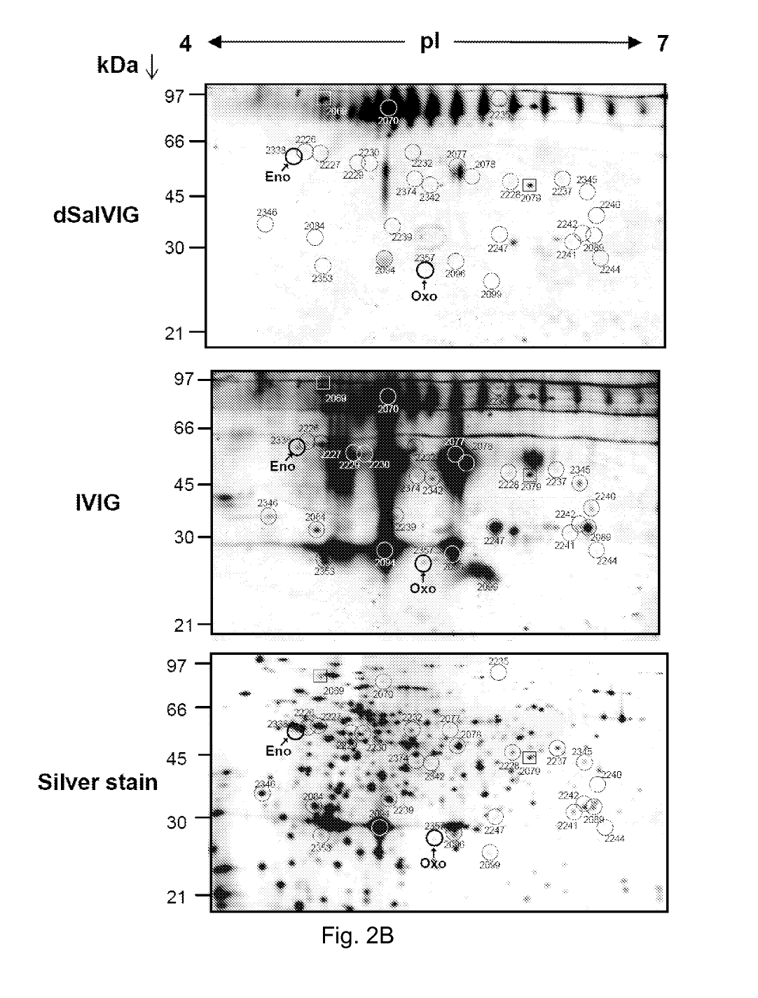

Detection and identification of immunogenic staphylococcal surface proteins by 2-DE analysis: In order to identify antigenic proteins, recognised by IgGs present in IVIG, cell wall associated proteins from S. aureus (clinical isolate ATCC 29213) were separated by 2-DE electrophoresis, followed by Western blot analysis using IVIG and dSaIVIG. To achieve high-quality spot resolution and to ensure adequate spot matching and identification, proteins were separated in two different pH ranges, pH 3-10 (FIG. 2A) and pH 4-7 (FIG. 2B), in series of three gels in parallel for each pH range. One of the gels was used for immunoblotting, probing the same blot twice, first with dSaIVIG (FIG. 2, top) and then IVIG (FIG. 2, middle). IVIG and dSaIVIG produced reproducibly distinct immunoblot profiles of 2-DE separated S. aureus surface proteins. The depletion of S. aureus-specific, opsonising IgGs by absorption of IVIG with S. aureus resulted in a considerably lower number of spots compared to immunoblots treated with IVIG (FIG. 2, top and middle). We assumed that proteins solely recognised by complete, but not with dSaIVIG may serve as vaccine candidates. Vice versa, proteinspots strongly reacting with both, IVIG and dSaIVIG, were supposed to be recognised by IgGs, that lack specificity for S. aureus antigens, and were therefore excluded from further investigation. For spot matching and identification, the two other gels were stained either with silver (FIG. 2, bottom) or coomassie (not shown). Spots of interest were cut from the coomassie stained gel and used for MALDI-TOF analysis. Eventually, about 40 proteins were identified using SUPRA (Table 1).

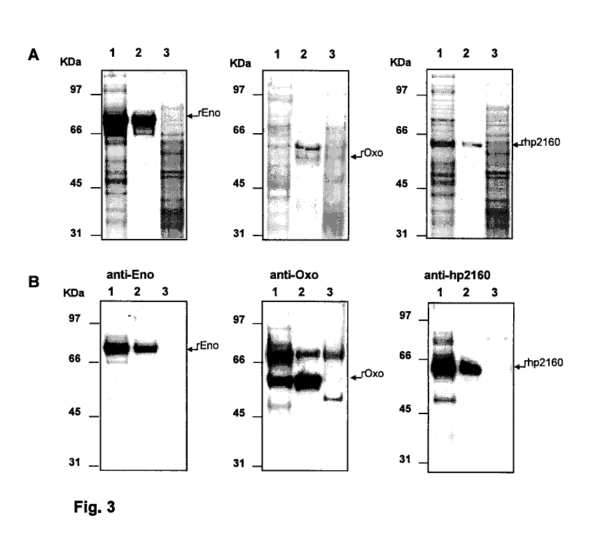

Expression of recombinant proteins and purification of specific antibodies: Three highly immunogenic proteins Enolase (Eno), oxoacyl-reductase (Oxo) and the hypothetical protein 2160 (hp2160) were repeatedly identified in 2-DE analysis. In order to prove their ability to trigger protective immune responses, these proteins were cloned, expressed as soluble GST-fusion proteins in E. coli and purified by affinity chromatography. The purity of the recombinant proteins was confirmed by SDS-PAGE (FIG. 3A).

To demonstrate the opsonic activity of antibodies against rEno, rOxo and rhp2160, corresponding antibodies from IVIG were purified by affinity chromatography. The specificity of enriched antibodies was analysed by immunoblotting. Western blots of purified antigens, cytoplasmic fractions of recombinant protein expressing E. coli as well as cytoplasmic fractions of non-transformed E. coli (BL21) were probed with affinity purified antibody fractions. As shown in FIG. 3B, each antibody fraction was able to recognise the corresponding antigen specifically: rEno (75.5 kDa), rOxo (54.5 kDa) and rhp2160 (64.0 kDa)

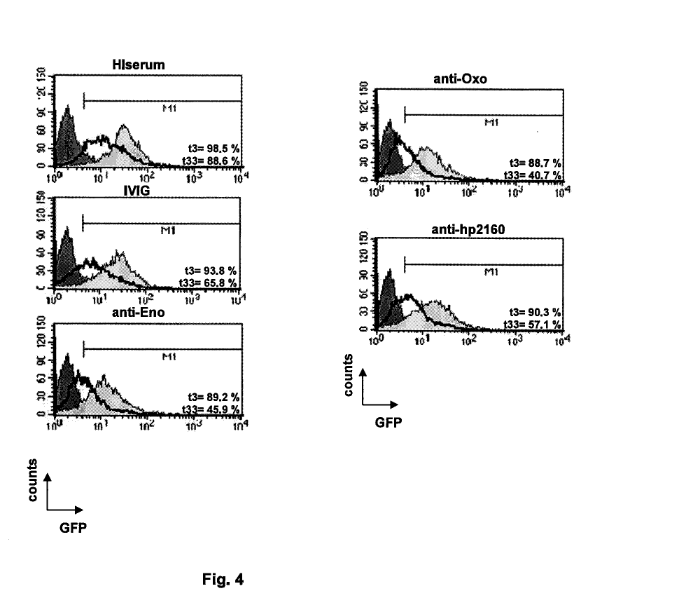

Opsonic effect of anti-Eno, -Oxo and -hp2160 antibodies: The opsonic activity of the affinity purified antibodies against rEno, rOxo and rhp2160 (anti-Eno, -Oxo and -hp2160) was analysed in a human PMNs-mediated opsonophagocytosis assay. The uptake of GFP-expressing S. aureus by human PMNs in presence of specific antibodies was monitored by flow cytometry. The opsonophagocytic activity mediated by the individual antibodies was compared to opsonophagocytic activity induced by IVIG and HIserum. As shown in FIG. 4, antibodies to any of the selected antigens were able to induce strong bacterial uptake by human PMNs. Within 3 min, 88-90% human PMNs stained positive for S. aureus indicating robust opsonising activity triggered by the affinity purified antibodies. Comparable phagocytosis rates were obtained for IVIG (˜93.8%) as well as for HIserum (98.5%). Phagocytosis was not observed in absence of IgGs.

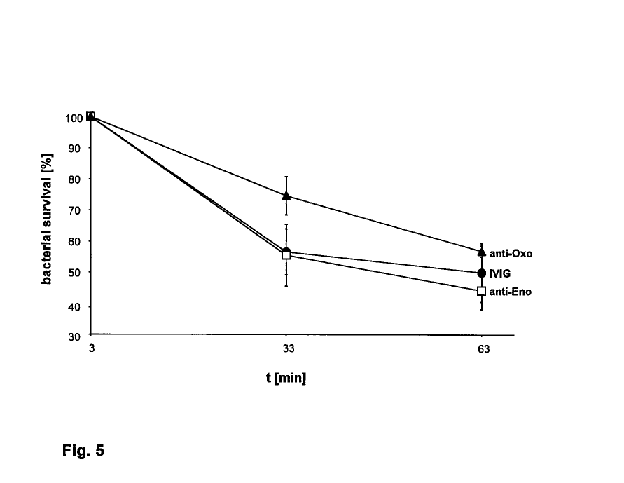

The percentage of GFP-positive human PMNs was significantly reduced in all samples, when measured 30 minutes after termination of phagocytosis (t33). To elucidate whether the reduction of green fluorescent PMNs was caused by the elimination of bacteria, viable intracellular S. aureus were quantified by CFU counting. As shown in FIG. 5, IVIG as well as specific antibodies are able to induce bacterial killing by PMNs. Using IVIG and anti-Eno as opsonins, 60% of the initial CFUs were recovered after 30 min (t33), further decreasing to 45% (anti-Eno) and 50% (IVIG) surviving bacteria after additional 30 min incubation (t63). When anti-Oxo served as opsonin, 75% of the initial CFU number was recovered at t33 and the PMNs mediated killing effect resulted in 58% surving bacteria 30 min later (t63).

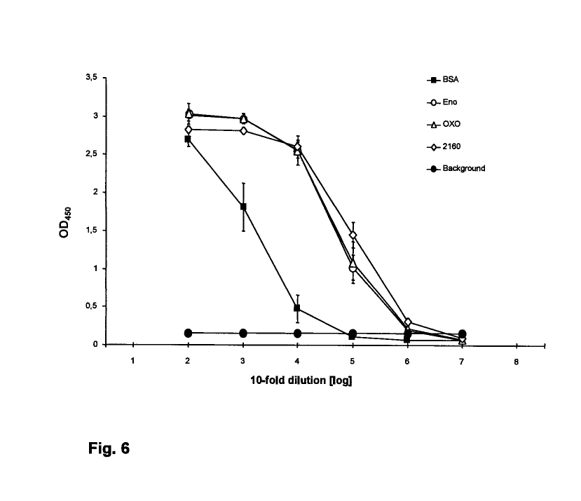

Immunisation with rEno, rOxo and rhp2160 induces significant levels of specific anti-S. aureus antibodies: Encouraged by the fact that anti-Eno, Oxo and hp2160 were able to induce opsonophagocytic killing of S. aureus by human PMNs in vitro, we used the recombinant proteins to induce a protective immune response to S. aureus infection in mice. Groups of 7 mice were immunised either with rEno, rOxo or rhp2160 and BSA as control. One week after the third administration of the booster injection the level of specific antibodies in serum was determined using ELISA. High levels of anti-Eno, -Oxo, -hp2160 and -BSA antibodies were detected in the serum of immunised mice. The anti-BSA antibody titer in mock-immunised mice was nearly 100-fold lower (FIG. 6).

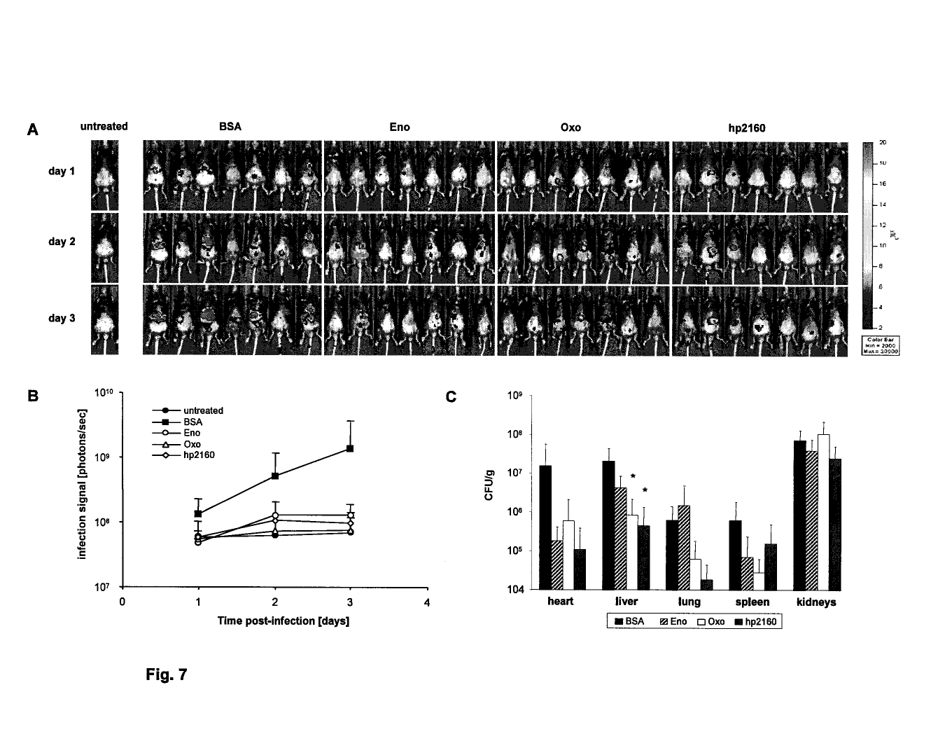

Protection of rEno, rOxo and rhp2160 immunised mice against S. aureus infection: To determine whether the immune response elicited by rEno, rOxo and rhp2160 was able to protect mice against S. aureus, immunised mice were challenged intravenously (i.v.) with the virulent, bioluminescent S. aureus Xen 29 strain with an inoculum of 10⁸ CFU. Mice were monitored by IVIS (in vivo imaging system) to follow the spreading of bacteria from day 1 to day 3 of infection. The in vivo bioluminescence images of the ventral side of an uninfected control, mock-immunised and recombinant protein-immunised mice are shown in FIG. 7A. Whereas a progressive increase of bioluminescence was observed in mice immunised with BSA, no significant increase of bioluminescence was detected in mice treated with rEno, rOxo or rhp2160. Quantitative analysis of emitted photons per second counted within the “region of interest” (ROI) for each imaged mouse is shown in FIG. 7B. Nearly constant bioluminescence values were observed in vaccinated mice over the course of infection, whereas a significant proliferation of bacteria, resulting in up to 10-fold increase of the bioluminescent signals, was measured in the group of mock-immunised mice.

In order to quantify the bacterial load in different organs by an independent method, CFUs of S. aureus in liver, kidneys, heart, lung and spleen were determined.

Vaccination with rEno, rOxo or rhp2160 resulted in remarkably lower S. aureus densities in all target tissues compared to the bacterial load in animals receiving BSA (FIG. 7C). The most obvious difference was observed in liver and heart tissue. In the liver the hp2160 immunised group revealed a 100-fold lower colonisation rate compared to the controls, followed by Oxo with a 10-fold lower bacterial load and finally Eno with 5-fold less bacteria. The in vivo data demonstrate that the immunisation with all three candidates (Eno, Oxo and hp2160) induces a protective immune response, which reduces an uncontrolled spread of S. aureus infection in mice.

TABLE 1
Cell wall-associated immunogenic antigens from S. aureus ATCC 29213 identified
by MALDI-TOF

| SEQ | | | | | | MASCOT^{c} | Seq. |
|---|---|---|---|---|---|---|---|
| ID | GenBank | | | | MW^{b} | Tot. | coverage^{d} |
| NO | Acc. No. | Spot ID | Putative identification | pI^{a} | (kDa) | score | (%) |
| 1 | gi\|15927930 | 2160 | esterase-like protein (hp2160) | 7.2 | 35.6 | 92 | 28 |
| 2 | gi\|15926453 | 2338 | enolase (Eno) | 4.6 | 47.1 | 206 | 47 |
| 3 | gi\|15926814 | 2357 | 3-oxoacyl-(acyl-carrier | 5.6 | 26.2 | 103 | 35 |
| | | | protein) reductase (Oxo) | | | | |
| 4 | gi\|15926530 | 2036/2078 | hypothetical protein, | 5.4 | 44.4 | 79 | 29 |
| 5 | gi\|15926396 | 2037/2038 | hypothetical protein, | 9.0 | 74.4 | 248 | 38 |
| 6 | gi\|2239274 | 20392345 | peptidoglycan hydrolase | 6.1 | 35.2 | 63 | 22 |
| 7 | gi\|15926560 | 2042 | conserved hypothetical | 5.6 | 31.8 | 121 | 48 |
| | | | protein | | | | |
| 8 | gi\|15928148 | 2043/2050/ | immunodominant antigen A | 6.1 | 24.2 | 80 | 23 |
| | | 2096 | | | | | |
| 9 | gi\|14247692 | 2053 | aldehyde dehydrogenase | 6.6 | 51.9 | 56 | 15 |
| 10 | gi\|21205110 | 2056 | truncated beta-hemolysin | 8.8 | 37.5 | 105 | 42 |
| 11 | gi\|15927879 | 2155 | secretory antigen precursor | 9.0 | 29.4 | 112 | 40 |
| | | | SsaA homolog | | | | |
| 12 | gi\|15927798 | 2158 | 50S ribosomal protein L13 | 9.3 | 16.3 | 228 | 66 |
| 13 | gi\|15926780 | 2161 | conserved hyp. protein | 5.7 | 34.6 | 186 | 45 |
| 14 | gi\|15926226 | 2219/2227 | translational elongation factor | 4.7 | 43.1 | 98 | 33 |
| | | | TU | | | | |
| 15 | gi\|15924043 | 2221 | autolysin | 9.7 | 102.6 | 77 | 11 |
| 16 | gi\|21205078 | 2222 | hypothetical protein | 5.8 | 65.5 | 63 | 13 |
| 17 | gi\|14247604 | 2406 | protoporphyrinogen oxidase | 6.3 | 52.2 | 126 | 23 |
| 18 | gi\|15927580 | 2425 | synergohymenotropic toxin | 8.6 | 38.7 | 78 | 14 |
| | | | precursor-like protein | | | | |
| 19 | gi\|6578924 | 2070 | glutamyl-t-RNAGln | 5.0 | 52.3 | 143 | 36 |
| 20 | gi\|15926504 | 2077 | aminotransferase NifS | 5.3 | 46.4 | 346 | 60 |
| | | | homolog | | | | |
| 21 | gi\|15926226 | 2084 | translational elongation factor | 4.4 | 29.6 | 153 | 29 |
| | | | TU | | | | |
| 22 | gi\|15923845 | 2089 | hypothetical protein | 5.7 | 29.4 | 113 | 38 |
| 23 | gi\|15928148 | 2094/2099 | immunodominant antigen A | 5.9 | 24.2 | 84 | 23 |
| 24 | gi\|15927677 | 2226 | ATP synthase beta chain | 4.7 | 51.4 | 317 | 55 |
| 25 | gi\|21204821 | 2228 | alanine dehydrogenase | 5.6 | 40.1 | 268 | 55 |
| 26 | gi\|15925843 | 2229/2230 | phosphopentomutase | 5.0 | 43.8 | 229 | 60 |
| 27 | gi\|15926547 | 2232 | NAD-specific glutamate | 5.2 | 45.9 | 255 | 50 |
| | | | dfehydrogenase | | | | |
| 28 | gi\|15928230 | 2235 | autolysin precursor-like | 6.0 | 69.2 | 72 | 16 |
| | | | protein | | | | |
| 29 | gi\|14247509 | 2237 | chorismate mutase homolog | 5.8 | 40.7 | 112 | 34 |
| 30 | gi\|15927670 | 2239 | SceD precursor-like protein | 5.5 | 24.1 | 70 | 34 |
| 31 | gi\|15923892 | 2240 | hypothetical protein | 6.5 | 37.1 | 89 | 19 |
| 32 | gi\|15926841 | 2241 | uridylate kinase | 6.0 | 26.3 | 88 | 26 |
| 33 | gi\|15926838 | 2242 | transcription pleiotropic | 5.9 | 28.7 | 178 | 49 |
| 34 | gi\|14247601 | 2244 | enterotoxin SEM | 6.5 | 27.5 | 68 | 28 |
| 35 | gi\|15926324 | 2247 | ferrichrome transport ATP- | 5.6 | 29.7 | 99 | 28 |
| 36 | gi\|15928275 | 2342 | conserved hypothetical | 5.3 | 37.5 | 188 | 33 |
| | | | protein | | | | |
| 37 | gi\|15926178 | 2346 | 50S ribosomal protein L25 | 4.4 | 23.8 | 104 | 34 |
| 38 | gi\|15926451 | 2353 | triosephosphate isomerase | 4.8 | 27.4 | 66 | 32 |
| 39 | gi\|13701328 | 2374 | Xaa-Pro dipeptidase | 5.2 | 39.6 | 241 | 36 |

^{a}Theoretical pI
^{b}Theoretical molecular weight (Da)
^{c}Identification probability of the fragment match
^{d}Sequence coverage of the total amino acid number

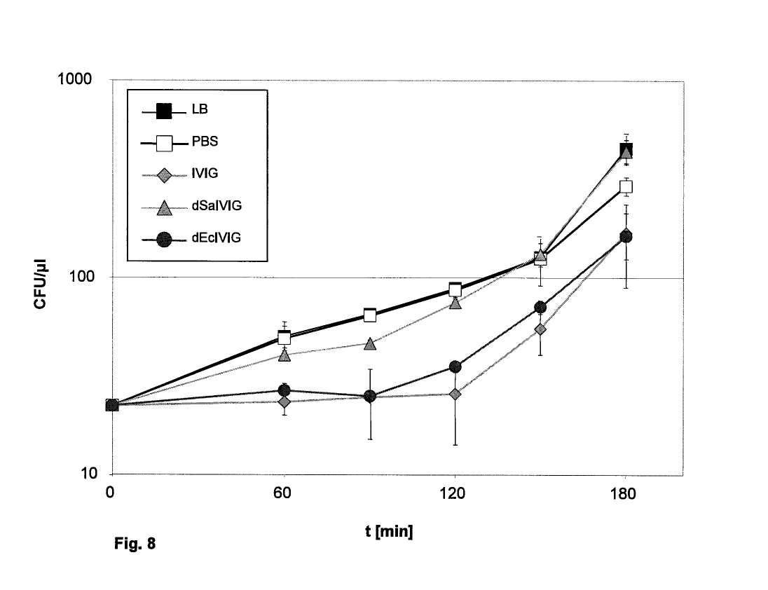

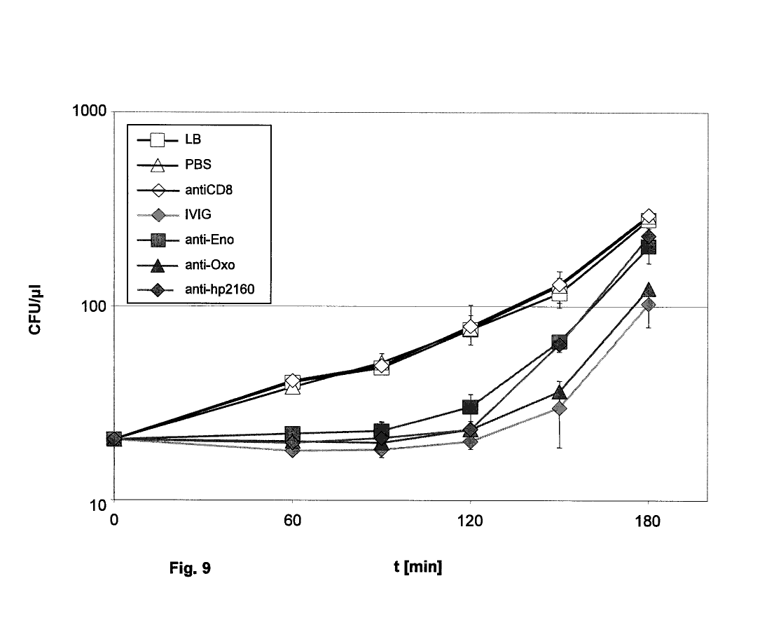

Specific IgGs exhibit a bacteriostatic effect on S. aureus growth in vitro: In order to analyse whether S. aureus specific IgGs could influence bacterial growth CFU numbers were compared over a period of three hours. As shown in FIG. 8 IVIG exhibits a bacteriostatic effect on S. aureus growth during the first two hours post inoculation. A comparable effect was observed when IVIG preabsorbed with E. coli (dEcIVIG) was used, whereas IVIG depleted of specific IgGs(dSaIVIG) did not influence the growth of S. aureus. These data indicate that the bacteriostatic effect is mediated exclusively by specific IgGs. In the next step enriched individual IgGs were analysed for their ability to influence bacterial growth. FIG. 9 shows that each of the specific IgGs inhibits the growth of S. aureus. Anti-Eno treated assays start growing after 90 min, whereas those treated with anti-Oxo are comparable to IVIG.

### Sequence Listing—Free Text

SEQ ID NOs: 1 to 39 S. aureus cell wall-associated proteins (Table 1)
SEQ ID NOs: 40 to 45 primer
SEQ ID NOs: 46 to 84 DNA sequences coding for SEQ ID NOs: 1-39

1. A pharmaceutical composition or a medicament comprising at least one cell wall-associated Staphylococcus aureus protein or a fragment or derivative thereof causing an immune response that induces opsonophagocytic activity of human neutrophils for S. aureus.

2. The pharmaceutical composition or medicament of claim 1, wherein the cell wall-associated protein
is derived from S. aureus strain ATCC 29213.

3. The pharmaceutical composition or medicament of claim 1, wherein the cell wall-associated protein is selected from the group consisting of the esterase-like protein (hp 2160) of SEQ ID NO: 1, the enolase (Eno) of SEQ ID NO: 2, the 3-oxoacyl-reductase (Oxo) of SEQ ID NO: 3, the NADH dehydrogenase like protein of SEQ ID NO: 4, the anion-binding protein like protein of SEQ ID NO: 5, the peptidoglycan hydrolase of SEQ ID NO: 6, the conserved hypothetical protein of SEQ ID NO: 7, the immunodominant antigen A of SEQ ID NO: 8, the aldehyde dehydrogenase of SEQ ID NO: 9, the truncated beta-hemolysin of SEQ ID NO: 10, the secretory antigen precursor SsaA homolog of SEQ ID NO: 11, the 50S ribosomal protein L13 of SEQ ID NO: 12, the conserved hypothetical protein of SEQ ID NO: 13, the translational elongation factor TU of SEQ ID NO: 14, the autolysin of SEQ ID NO: 15, the hypothetical protein of SEQ ID NO: 16, the protoporphyrinogen oxidase of SEQ ID NO: 17, synergohymenotropic toxin precursor-like protein of SEQ ID NO: 18, the glutamyl-t-RNAGln amidotransferase subunit A of SEQ ID NO: 19, the aminotransferase NifS homologue of SEQ ID NO: 20, the translational elongation factor TU of SEQ ID NO: 21, the hypothetical protein of SEQ ID NO: 22, the immunodominant antigen A of SEQ ID NO: 23, the ATP synthase beta chain of SEQ ID NO: 24, the alanine dehydrogenase of SEQ ID NO: 25, the phosphopentomutase of SEQ ID NO: 26, the NAD-specific glutamate dehydrogenase of SEQ ID NO: 27, autolysin precursor-like protein of SEQ ID NO: 28, the chorismate mutase homolog of SEQ ID NO: 29, the SceD precursor-like protein of SEQ ID NO: 30, the hypothetical protein of SEQ ID NO: 31, the uridylate kinase of SEQ ID NO: 32, the transcription pleiotropic repressor codY of SEQ ID NO: 33, the enterotoxin SEM of SEQ ID NO: 34, the ferrichrome transport ATP-binding protein of SEQ ID NO: 35, the conserved hypothetical protein of SEQ ID NO: 36, the 50S ribosomal protein L25 of SEQ ID NO: 37, the triosephosphate isomerase of SEQ ID NO: 38, the Xaa-Pro dipeptidase of SEQ ID NO: 39 and fragments and derivatives thereof.

4. The pharmaceutical composition or medicament of claim 1,
wherein the fragments comprises at least 10 amino acids.

5. (canceled)

6. A cell wall-associated Staphylococcus aureus protein selected from the group consisting of SEQ ID NOs: 1, 3-5, 7, 9, 11-13, 16-18, 20, 22, 28-32, 36-39 and fragments and derivatives thereof.

7. An antibody against a protein as defined in claim 6.

8. A diagnostic reagent, a pharmaceutical composition or a medicament comprising at least one antibody as defined in claim 7 or an antibody against the protein defined in claim 3.

9. A DNA sequence encoding the wall-associated S. aureus protein or a fragment or derivative thereof as defined in claim 6.

10. A vector carrying the DNA of claim 9.

11. A host cell transformed with the vector of claim 10 and/or carrying the DNA of claim 9.

12. A method for producing the cell wall-associated S. aureus protein or a fragment or derivative thereof as defined in claim 6, which method comprises culturing the host cell of claim 11.

13. (canceled)

14. A method for vaccinating a human or animal against Staphylococcus aureus which comprises administering the human or animal with cell wall-associated S. aureus protein or a fragment or derivative thereof as defined in claim 1.

15. A method for treatment of a Staphylococcus aureus infection in a human or animal which comprises administering the human or animal at least one antibody of claim 7 or an antibody against the protein defined in claim 3.

16. The pharmaceutical composition or medicament of claim 1, wherein the cell wall-associated protein solely reacts with an intravenous immunoglobulin (IVIG) preparation but not with an IVIG preparation depleted of S. aureus-specific opsonising antibodies.

17. The pharmaceutical composition or medicament of claim 3, wherein the cell wall-associated protein is selected from the group consisting of the Eno of SEQ ID NO: 2, the Oxo of SEQ ID NO: 3, hp2160 of SEQ ID NO: 1 and fragments and derivatives thereof.

18. The pharmaceutical composition or medicament of claim 1, wherein the derivatives is selected from the group consisting of modifications of functional groups, linkage of functional groups, linkage to at least one further functional protein domain and linkage to other biologically active molecules.

19. The pharmaceutical composition or medicament of claim 1, which is a protective Staphylococcus aureus vaccine.


SEQUENCE LISTING (84 sequences)

SEQ ID NO: 1 - protein, 306 aa, Staphylococcus aureus
MIRNRVMNSVVNKYLLHNRSIMFKNDQDVERFFYKREIENRKKHKQPSTLNVKANLEKLSLDDMQVFRFNFRHQIDKKILYIHGGFNALQPSPFHWRLLDKITLSTLYEVVLPIYPKTPEFHIDDTFQAIQRVYDQLVSEVGHQNVVVMGDGSGGALALSFVQSLLDNQQPLPNKLYLISPILDATLSNKDISDALIEQDAVLSQFGVNEIMKKWANGLPLTDKRISPINGTIEGLPPVYMFGGGREMTHPDMKLFEQMMLQHHQYIEFYDYPKMVHDFPIYPIRQSHKAIKQIAKSIDEDVTQNN

SEQ ID NO: 2 - protein, 434 aa, Staphylococcus aureus [sequence omitted]

SEQ ID NO: 3 - protein, 246 aa, Staphylococcus aureus
MKMTKSALVTGASRGIGRSIALQLAEEGYNVAVNYAGSKEKAEAVVEEIKAKGVDSFAIQANVADADEVKAMIKEVVSQFGSLDVLVNNAGITRDNLLMRMKEQEWDDVIDTNLKGVFNCIQKATPQMLRQRSGAIINLSSVVGAVGNPGQANYVATKAGVIGLTKSAARELASRGITVNAVAPGFIVSDMTDALSDELKEQMLTQIPLARFGQDTDIANTVAFLASDKAKYITGQTIHVNGGMYM

SEQ ID NO: 4 - protein, 402 aa, Staphylococcus aureus
MAQDRKKVLVLGAGYAGLQTVTKLQKAISTEEAEITLINKNEYHYEATWLHEASAGTLNYEDVLYPVESVLKKDKVNFVQAEVTKIDRDAKKVETNQGIYDFDILVVALGFVSETFGIEGMKDHAFQIENVITARELSRHIEDKFANYAASKEKDDNDLSILVGGAGFTGVEFLGELTDRIPELCSKYGVDQNKVKITCVEAAPKMLPMFSEELVNHAVSYLEDRGVEFKIATPIVACNEKGFVVEVDGEKQQLNAGTSVWAAGVRGSKLMEESFEGVKRGRIVTKQDLTINGYDNIFVIGDCSAFIPAGEERPLPTTAQIAMQQGESVAKNIKRILNGESTEEFEYVDRGTVCSLGSHDGVGMVFGKPIAGKKAAFMKKVIDTRAVFKIGGIGLAFKKGKF

SEQ ID NO: 5 - protein, 646 aa, Staphylococcus aureus
MSSQKKKISLFAFFLLTVITITLKTYFSYYVDFSLGVKGLVQNLILLMNPYSLVALVLSVFLFFKGKKAFWFMFIGGFLLTFLLYANVVYFRFFSDFLTFSTLNQVGNVESMGGAVSASFKWYDFVYFIDTLVYLFILIFKTKWLDTKAFSKKFVPVVMAASVALFFLNLAFAETDRPELLTRTFDHKYLVKYLGPYNFTVYDGVKTIENNQQKALASEDDLTKVLNYTKQRQTEPNPEYYGVAKKKNIIKIHLESFQTFLINKKVNGKEVTPFLNKLSSGKEQFTYFPNFFHQTGQGKTSDSEFTMDNSLYGLPQGSAFSLKGDNTYQSLPAILDQKQGYKSDVMHGDYKTFWNRDQVYKHFGIDKFYDATYYDMSDKNVVNLGLKDKIFFKDSANYQAKMKSPFYSHLITLTNHYPFTLDEKDATIEKSNTGDATVDGYIQTARYLDEALEEYINDLKKKGLYDNSVIMIYGDHYGISENHNNAMEKLLGEKITPAKFTDLNRTGFWIKIPGKSGGINNEYAGQVDVMPTILHLAGIDTKNYLMFGTDLFSKGHNQVVPFRNGDFITKDYKYVNGKIYSNKNNELITTQPADFEKNKKQVEKDLEMSDNVLNGDLFRFYKNPDFKKVNPSKYKYETGPKANSKK

SEQ ID NO: 6 - protein, 322 aa, Staphylococcus aureus
MEDVLYMKKLTAAAIATMGFATFTMAHQADSAETTNTQQAHTQMSTQSQDVSYGTYYTIDSNGDYHHTPDGNWNQAMFDNKEYSYTFVDAQGHTHYFYNCYPKNANANGSGQTYVNPATAGDNNDYTASQSQQHINQYGYQSNVGPDASYYSHSNNNQAYNSHDGNGKVNYPNGTSNQNGGSASKATRSGHAKDASWLTSRKQLQPYGQYHGGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSR

SEQ ID NO: 7 - protein, 274 aa, Staphylococcus aureus
MQPHLICLDLDGTLLNDNKEISSYTKQVLNELQQRGHQIMIATGRPYRASQMYYHELNLTTPIVNFNGAYVHHPKDKNFKTCHEILDLGIAQNIIQGLQQYQVSNIIAEVKDYVFINNHDPRLFEGFSMGNPRIQTGNLLVHLKESPTSILIEAEESKIPEIKNMLTHFYADHIEHRRWGAPFPVIEIVKLGINKARGIEQVRQFLNIDRNNIIAFGDEDNDIEMIEYARHGVAMENGLQELKDVANNITFNNNEDGIGRYLNDFFNLNIRYYC

SEQ ID NO: 8 - protein, 233 aa, Staphylococcus aureus
MKKTIMASSLAVALGVTGYAAGTGHQAHAAEVNVDQAHLVDLAHNHQDQLNAAPIKDGAYDIHFVKDGFQYNFTSNGTTWSWSYEAANGQTAGFSNVAGADYTTSYNQGSDVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 9 - protein, 459 aa, Staphylococcus aureus
MNIIEQKFYDSKAFFNTQQTKDISFRKDQLKKLSKAIKSYESDILEALYTDLGKNKVEAYATEIGITLKSIKNARKELKNWTKTKNVDTPLYLFPTKSYIKKEPYGTVLIIAPFNYPFQLVFEPLIGAIAAGNTAIIKPSELTPNVARVIKRLINETFDANYIEVIEGGIEETQTLIHLPFDYVFFTGSENVGKIVYQAASENLVPVTLEMGGKSPVIVDETANIKVASERICFGKFTNAGQTCVAPDYILVHESVKDDLITALSKTLREFYGQNIQQSPDYGRIVNLKHYHRLTSLLNSAQMNIVFGGHSDEDERYIEPTLLDHVTSDSAIMQEEIFGPILPILTYQSLDEAIAFIHQRPKPLSLYLFSEDENATQRVINELSFGGGAINDTLMHLANPKLPFGGVGASGMGRYHGKYSFDTFTHEKSYIFKSTRLESGVHLPPYKGKFKYIKAFFKN

SEQ ID NO: 10 - protein, 274 aa, Staphylococcus aureus
MYPNWGQYKRADLIGQSSYIKNNDVVIFNEAFDNGASDKLLSNVKKEYPYQTPVLGRSQSGWDKTEGSYSSTVAEDGGVAIVSKYPIKEKIQHVFKSGCGFDNDSNKGFVYTKIEKNGKNVHVIGTHTQSEDSRCGAGHDRKIRAEQMKEISDFVKKKNIPKDETVYIGGDLNVNKGTPEFKDMLKNLNVNDVLYAGHNSTWDPQSNSIAKYNYPNGKPEHLDYIFTDKDHKQPKQLVNEVVTEKPKPWDVYAFPYYYVYNDFSDHYPIKAYSK

SEQ ID NO: 11 - protein, 267 aa, Staphylococcus aureus
MKKIATATIATAGFATIAIASGNQAHASEQDNYGYNPNDPTSYSYTYTIDAQGNYHYTWKGNWHPSQLNQDNGYYSYYYYNGYNNYNNYNNGYSYNNYSRYNNYSNNNQSYNYNNYNSYNTNSYRTGGLGASYSTSSNNVQVTTTMAPSSNGRSISSGYTSGRNLYTSGQCTYYVFDRVGGKIGSTWGNASNWANAAARAGYTVNNTPKAGAIMQTTQGAYGHVAYVESVNSNGSVRVSEMNYGYGPGVVTSRTISASQAAGYNFIH

SEQ ID NO: 12 - protein, 145 aa, Staphylococcus aureus
MRQTFMANESNIERKWYVIDAEGQTLGRLSSEVASILRGKNKVTYTPHVDTGDYVIVINASKIEFTGNKETDKVYYRHSNHPGGIKSITAGELRRTNPERLIENSIKGMLPSTRLGEKQGKKLFVYGGAEHPHAAQQPENYELRG

SEQ ID NO: 13 - protein, 305 aa, Staphylococcus aureus
METYEFNITDKEQTGMRVDKLLPELNNDWSRNQIQDWIKAGLVVANDKVVKSNYKVKLNDHIVVTEKEVVEADILPENLNLDIYYEDDDVAVVYKPKGMVVHPSPGHYTNTLVNGLMYQIKNLSGINGEIRPGIVHRIDMDTSGLLMVAKNDIAHRGLVEQLMDKSVKRKYIALVHGNIPHDYGTIDAPIGRNKNDRQSMAVVDDGKEAVTHFNVLEHFKDYTLVECQLETGRTHQIRVHMKYIGFPLVGDPKYGPKKTLDIGGQALHAGLIGFEHPVTGEYIERHAELPQDFEDLLDTIRKRDA

SEQ ID NO: 14 - protein, 394 aa, Staphylococcus aureus
MAKEKFDRSKEHANIGTIGHVDHGKTTLTAAIATVLAKNGDSVAQSYDMIDNAPEEKERGITINTSHIEYQTDKRHYAHVDCPGHADYVKNMITGAAQMDGGILVVSAADGPMPQTREHILLSRNVGVPALVVFLNKVDMVDDEELLELVEMEVRDLLSEYDFPGDDVPVIAGSALKALEGDAQYEEKILELMEAVDTYIPTPERDSDKPFMMPVEDVFSITGRGTVATGRVERGQIKVGEEVEIIGLHDTSKTTVTGVEMFRKLLDYAEAGDNIGALLRGVAREDVQRGQVLAAPGSITPHTEFKAEVYVLSKDEGGRHTPFFSNYRPQFYFRTTDVTGVVHLPEGTEMVMPGDNVEMTVELIAPIAIEDGTRFSIREGGRTVGSGVVTEIIK

SEQ ID NO: 15 - protein, 943 aa, Staphylococcus aureus
MLGVINRMAKKFNYKLPSMVALTLVGSAVTAHQVQAAETTQDQTTNKNVLDSNKVKATTEQAKAEVKNPTQNISGTQVYQDPAIVQPKTANNKTGNAQVSQKVDTAQVNGDTRANQSATTNNTQPVAKSTSTTAPKTNTNVTNAGYSLVDDEDDNSEHQINPELIKSAAKPAALETQYKAAAPKAKTEATPKVTTFSASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQFTTTPTTPSKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKSGTKLYTVPWGTSKQVAGSVSGSGNQTFKASKQQQIDKSIYLYGSVNGKSGWVSKAYLVDTAKPTPTPIPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYTVTKPQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYYTVLLITALVGKAKDLPHQLGNQLHQLPKI

SEQ ID NO: 16 - protein, 564 aa, Staphylococcus aureus
MTDYVTKYAKKVVSGEILASLKNIQVCKRHLSFMENPPNGCHWDNHLSNKAIKFVEMLPDPKTNQPMPLMEFQKFIVGSLYGWRRGQYRMFTKAYISMARKQGKSLIVSGMSVNELLFGQYPKFNRQIYVASSTYKQAQTIFKMASQQVNLMRSKSKFIREKTDVRKTDIEDVLSSSVFAPLSNNPDAVDGKDPTVAILDELASMPDDEMYSRFKTGMTLQKNPLTLLVSTAGDNLNSQMYQEYKYIKRILNEEVRADNYFVYCAEMDSQEEVQDETKWIKAMPLLESKEHRKTILQNVKADIQDELEKGTSYHKILIKNFNLWQAQREDSLLDISDWEQVITPMPNINGKDVYIGVDLSRLDDLTSVGFIFPNDDKKVFLHSHSFIGLRTNLEQKSKRDKINYELAIERGEAETTQSDSGMIDYKQVIDFIVKFITTHDLNVQAVCYDPWNAQSFITTIESMALDWPLIEVGQSFKALSQSIKEFRMWVADERIQHNDNMLLTTSVNNAVLIRDGEDNVKINKKMNRQKIDPIISIITAFTEARMHEFQENWTEKYESEEFGF

SEQ ID NO: 17 - protein, 466 aa, Staphylococcus aureus
MTKSVAIIGAGITGLSSAYFLKQQDPNIDVTIFEASNRPGGKIQSYRKDGYMIELGPESYLGRKTIMTELAKDIGLEQDIVTNTTGQSYIFAKNKLYPIPGGSIMGIPTDIKPFVTTKLISPLGKLRAGLDLIKKPIQMQDGDISVGAFFRARLGNEVLENLIEPLMGGIYGTDIDKLSLMSTFPNFKEKEEAFGSLIKGMKDEKNKRLKQRQLYPGAPKGQFKQFKHGLSSFIEALEQDVKNKGVTIRYNTSVDDIITSQKQYKIVYSNQQEDVFDGVLVTTPHQVFLNWFGQDPAFDYFKTMDSTTVATVVLAFDEKDIENTYDGTGFVIARTSDTDITACTWTSKKWPFTTPEGKVLIRAYVGKPGDTVVDDHTDNELVSIVRRDLSQMMTFKGDPEFTIVNRLPKSMPQYHVGHIQQIRQIQAHIKQTYPRLRVTGASFEAVGLPDCITQGKVAAEEVIAEL

SEQ ID NO: 18 - protein, 338 aa, Staphylococcus aureus
MIKQLYKNITICSLAISTALTVFPATSYAKINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 19 - protein, 485 aa, Staphylococcus aureus
MSIRYESVENLLTLIKDKKIKPSDVVKDIYDAIEETDPTIKSFLALDKENAIKKAQELDELQAKDQMDGKLFGIPMGIKDNIITNGLETTCASKMLEGFVPIYESTVMEKLHNENAVLIGKLNMDEFAMGGSTETSISKKTVNPFDHKAVPGGSSGGSAAAVAAGLVPFSLGSDTGGSIRQPAAYCGVVGMKPTYGRVSRFGLVAFASSLDQIGPLTRNVKDNAIVLEAISGADANDSTSAPVDDVDFTSEIGKDIKGLKVALPKEYLGEGVADDVKEAVQNAVETLKSLGAVVEEVSLPNTKFGIPSYYVIASSEASSNLSRFDGIRYGYHSKEAHSLEELYKMSRSEGFGKEVKRRIFLGTFALSSGYYDAYYKKSQKVRTLIKNDFDKVFENYDVVVGPTAPTTAFNLGEEIDDPLTMYANDLLTTPVNLAGLPGISVPCGQSNGRPIGLQFIGKPFDEKTLYRVAYQYETQYNLHDVYEKL

SEQ ID NO: 20 - protein, 413 aa, Staphylococcus aureus
MAEHSFDVNEVIKDFPILDQKVNGKRLAYLDSTATSQTPMQVLNVLEDYYKRYNSNVHRGVHTLGSLATDGYENARETVRRFINAKYFEEIIFTRGTTASINLVAHSYGDANVEEGDEIVVTEMEHHANIVPWQQLAKRKNATLKFIPMTADGELNIEDIKQTINDKTKIVAIAHISNVLGTINDVKTIAEIAHQHGAIISVDGAQAAPHMKLDMQEMNADFYSFSGHKMLGPTGIGVLFGKRELLQKMEPIEFGGDMIDFVSKYDATWADLPTKFEAGTPLIAQAIGLAEAIRYLERIGFDAIHKYEQELTIYAYEQMSAIEGIEIYGPPKDRRAGVITFNLQDVHPHDVATAVDTEGVAVRAGHHCAQPLMKWLNVSSTARASFYIYNTKEDIDQLINALKQTKEFFSYEF

SEQ ID NO: 21 - protein, 394 aa, Staphylococcus aureus; identical to SEQ ID NO: 14

SEQ ID NO: 22 - protein, 259 aa, Staphylococcus aureus
MQALQTFNFEELPVRTLEVDGEPYFIGKDVADILGYANGRDALSKHVDEDDKKVLTSRNTTLENLPNRGLTAVNESGLYSLIFSSKLESAKRFKRWVTSDVLPAIRKYGIYATDNVIEQTLKDPDYIITVLTEYKKEKEQNLLLQQEIGELKPKADYVDEILKSTGTLATTQIAADYGISAQKLNKLLHEARLQRKVNKQWVLYSEHMGKSYTDSDTITIVRSDGREDTVLQTRWTQKGRLKIHEIMTEFGYEANLGGA

SEQ ID NO: 23 - protein, 233 aa, Staphylococcus aureus; identical to SEQ ID NO: 8

SEQ ID NO: 24 - protein, 470 aa, Staphylococcus aureus
MGIGRVTQVMGPVIDVRFEHNEVPKINNALVIDVPKEEGTIQLTLEVALQLGDDVVRTIAMDSTDGVQRGMDVKDTGKEISVPVGDETLGRVFNVLGETIDLKEEISDSVRRDPIHRQAPAFDELSTEVQILETGIKVVDLLAPYIKGGKIGLFGGAGVGKTVLIQELINNIAQEHGGISVFAGVGERTREGNDLYFEMSDSGVIKKTAMVFGQMNEPPGARMRVALSGLTMAEYFRDEQGQDVLLFIDNIFRFTQAGSEVSALLGRMPSAVGYQPTLATEMGQLQERITSTTKGSVTSIQAVFVPADDYTDPAPATAFAHLDATTNLERKLTEMGIYPAVDPLASTSRALEPSIVGQEHYEVARDVQSTLQKYRELQDIIAILGMDELSDEDKQTVERARRIQFFLSQNFHVAEQFTGQKGSYVPVKTTVANFKDILDGKYDHIPEDAFRLVGSMDDVIAKAKDMGVEV

SEQ ID NO: 25 - protein, 372 aa, Staphylococcus aureus
MKIGIPREIKNNENRVGLSPSGVHALVESGHTVLVETNAGSGSFFEDVDYKEAGAEIVAEQAKVWDVDMVIKVKEPLESEYPYFKEGLVLFTYLHLANEEKLTQALIDRKVISIAYETVQLPDRSLPLLSPMSEVAGRMSAQVGAEFLQKLNGGMGILLGGVPGVPKGKVTIIGGGQAGTNAAKIALGLGADVTILDVNPKRLQQLDDLFGGRVHTIMSNPLNIELYVKQSDLVIGAVLIPGAKAPRLVTEDMIKQMKNGSVIIDIAIDQGGIFETTDKITTHDDPTYIKHGVVHYAVANMPGAVPRTSTLALNNATLPYALMLANKGYREAFKSNQPLSLGLNTYKGHVTNKGVAEAFEMEYKSVEEALQL

SEQ ID NO: 26 - protein, 392 aa, Staphylococcus aureus
MTRPFNRVHLIVMDSVGIGEAPDAADFKDEGSHTLRHTLEGFDQTLPNLEKLGLGNIDKLPVVNAVEQPEAYYTKLSEASVGKDTMTGHWEIMGLNIMQPFKVYPNGFPEELIQQIEEMTGRKVVANKPASGTQIIDEWGEHQMKTGDLIVYTSADPVLQIAAHEDIIPLEELYDICEKVRELTKDPKYLIGRIIARPYVGEPGNFTRTSNRHDYALKPFGKTVLDHLKDGGYDVIAIGKINDIYDGEGVTEAVRTKSNMDGMDQLMKIVKKDFTGISFLNLVDFDALYGHRRDKPGYAQAIKDFDDRLPELFSNLKEDDLVIITADHGNDPTAPGTDHTREYIPVIMYSPKFKGGHALESDTTFSSIGATIADNFNVTLPEFGKSYLKELK

SEQ ID NO: 27 - protein, 414 aa, Staphylococcus aureus
MTENNNLVTSTQGIIKEALHKLGFDEGMYDLIKEPLRMLQVRIPVRMDDGTVKTFTGYRAQHNDAVGPTKGGVRFHPDVDEEEVKALSMWMTLKCGIVNLPYGGGKGGIVCDPRQMSIHEVERLSRGYVRAISQFVGPNKDIPAPDVFTNSQIMAWMMDEYSALDKFNSPGFITGKPIVLGGSHGRDRSTALGVVIAIEQAAKRRNMQIEGAKVVIQGFGNAGSFLAKFLYDLGAKIVGISDAYGALHDPNGLDIDYLLDRRDSFGTVTNLFEETISNKELFELDCDILVPAAISNQITEDNAHDIKASIVVEAANGPTTPEATRILTERGILLVPDVLASAGGVTVSYFEWVQNNQGYYWSEEEVNEKLREKLEAAFDTIYELSQNRKIDMRLAAYIIGIKRTAEAARYRGWA

SEQ ID NO: 28 - protein, 619 aa, Staphylococcus aureus
MPKNKILIYLLSTTLVLPTLVSPTAYADTPQKDTTAKTTSHDSKKSNDDETSKDTTSKDTDKADNNNTSNQDNNDKKFKTIDDSTSDSNNIIDFIYKNLPQTNINQLLTKNKYDDNYSLTTLIQNLFNLNSDISDYEQPRNGEKSTNDSNKNSDNSIKNDTDTQSSKQDKADNQKAPKSNNTKPSTSNKQPNSPKPTQPNQSNSQPASDDKANQKSSSKDNQSMSDSALDSILDQYSEDAKKTQKDYASQSKKDKNEKSNTKNPQLPTQDELKHKSKPAQSFNNDVNQKDTRATSLFETDPSISNNDDSGQFNVVDSKDTRQFVKSIAKDAHRIGQDNDIYASVMIAQAILESDSGRSALAKSPNHNLFGIKGAFEGNSVPFNTLEADGNKLYSINAGFRKYPSTKESLKDYSDLIKNGIDGNRTIYKPTWKSEADSYKDATSHLSKTYATDPNYAKKLNSIIKHYQLTQFDDERMPDLDKYERSIKDYDDSSDEFKPFREVSDSMPYPHGQCTWYVYNRMKQFGTSISGDLGDAHNWNNRAQYRDYQVSHTPKRHAAVVFEAGQFGADQHYGHVAFVEKVNSDGSIVISESNVKGLGIISHRTINAAAAEELSYITGK

SEQ ID NO: 29 - protein, 363 aa, Staphylococcus aureus
MSNKLESYRSEIVSLNHQILDLLSKRGELAQKIGEEKLKQGTRIYDPQREKEMLNDLIDSNKGPFNDNTIKQLFKEIFKASTDLQKSENEKHLYVSRKLKPEDTIVTFDNGGIIGDGNKSFVFGPCSVESFEQVEAVAKNLHAKGEKFIRGGAFKPRTSPYDFQGLGVEGLKILKQIKDKYDLNVVSEIVNPNDFEVADEYLDVFQIGARNMQNFELLKEAGRTKKPILLKRGLSATIEEFVYAAEYIASQGNQNIILCERGIRTYEKATRNTLDISAVPILKQGTHLPVMVDVTHSTGRKDIMLPTAKAALAVGADGVMAEVHPDPSVALSDAGQQMDLDEFQAFYDELKPLADLYNAKKLK

SEQ ID NO: 30 - protein, 231 aa, Staphylococcus aureus
MKKTLLASSLAVGLGIVAGNAGHEAHASEADLNKASLAQMAQSNDQTLNQKPIEAGAYNYTFDYEGFTYHFESDGTHFAWNYHATGANGANMSAQAPATNNVEPSAVQANQVQSQEVEAPQNAQTQQPQASTSNNSQVTATPTESKASEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 31 - protein, 315 aa, Staphylococcus aureus
MDIELTKKDGTVIKLSEYGFIVNDIVIDSMQINTKYQDKENMNGRILMGSNYISRDIVVPCFCVVKNRSDIAYMRDMLYSLTTDIEPMYLREIRRKEELNYRFTQPISDDYVKLDKNNFPDYEYSRHDQQNYVNGKQYKVIFNGVINPKQKGNKVSFELKFETTELPYGESIGTSLELEENKKVGLWSFDFNIDWHAGGDKRKYTFENLSKGTVYYHGSAPNDQFNMYKKITIILGEDTESFVWNLTHAEIMKIEGIKLKTGDRIVYDSFRVYKNGVEISTETNIAQPKFKYGANKFEFNQTVQKVQFDLKFYYK

SEQ ID NO: 32 - protein, 240 aa, Staphylococcus aureus
MAQISKYKRVVLKLSGEALAGEKGFGINPVIIKSVAEQVAEVAKMDCEIAVIVGGGNIWRGKTGSDLGMDRGTADYMGMLATVMNALALQDSLEQLDCDTRVLTSIEMKQVAEPYIRRRAIRHLEKKRVVIFAAGIGNPYFSTDTTAALRAAEVEADVILMGKNNVDGVYSADPKVNKDAVKYEHLTHIQMLQEGLQVMDSTASSFCMDNNIPLTVFSIMEEGNIKRAVMGEKIGTLITK

SEQ ID NO: 33 - protein, 257 aa, Staphylococcus aureus
MSLLSKTRELNTLLQKHKGIAVDFKDVAQTISSVTVTNVFIVSRRGKILGSSLNELLKSQRIIQMLEERHIPSEYTERLMEVKQTESNIDIDNVLTVFPPENRELFIDSRTTIFPILGGGERLGTLVLGRVHDDFNENDLVLGEYAATVIGMEILREKHSEVEKEARDKAAITMAINSLSYSEKEAIEHIFEELGGTEGLLIASKVADRVGITRSVIVNALRKLESAGVIESRSLGMKGTFIKVKKEKFLDELEKSK

SEQ ID NO: 34 - protein, 239 aa, Staphylococcus aureus
MKRILIIVVLLFCYSQNHIATADVGVLNLRNYYGSYPIEDHQSINPENNHLSHQLVFSMDNSTVTAEFKNVDDVKKFKNHAVDVYGLSYSGYCLKNKYIYGGVTLAGDYLEKSRRIPINLWVNGEHQTISTDKVSTNKKLVTAQEIDTKLRRYLQEEYNIYGFNDTNKGRNYGNKSKFSSGFNAGKILFHLNDGSSFSYDLFDTGTGQAESFLKIYNDNKTVETEKFHLDVEISYKDES

SEQ ID NO: 35 - protein, 265 aa, Staphylococcus aureus
MNRLHGQQVKIGYGDNTIINKLDVEIPDGKVTSIIGPNGCGKSTLLKALSRLLAVKEGEVFLDGENIHTQSTKEIAKKIAILPQSPEVADGLTVGELVSYGRFPHQKGFGRLTAEDKKEIDWAMEVTGTDTFRHRSINDLSGGQRQRVWIAMALAQRTDIIFLDEPTTYLDICHQLEILELVQKLNQEQGCTIVMVLHDINQAIRFSDHLIAMKEGDIIATGSTEDVLTQEILEKVFNIDVVLSKDPKTGKPLLVTYDLCRRAYS

SEQ ID NO: 36 - protein, 318 aa, Staphylococcus aureus
MNYQVLLYYKYTTIDDPEQFAQDHLAFCKAHHLKGRILVSTEGINGTLSGTKEETEQYMAHMHADERFKDMVFKIDEAEGHAFKKMHVRPRKEIVALDLEDDVDPRHTTGQYLSPVEFRKALEDDDTVIIDARNDYEFDLGHFRGAIRPDITRFRDLPDWIKENKALFTDKKVVTYCTGGIRCEKFSGWLLKEGFEDVAQLHGGIATYGKDPETKGQYWDGKMYVFDDRISVDINQVEKTIIGKDWFDGKPCERYINCANPECNKQILVSEENETKYLGACSYECAKHERNRYVQANNISDNEWQQRLTNFDDLHQHA

SEQ ID NO: 37 - protein, 217 aa, Staphylococcus aureus
MASLKSIIRQGKQTRSDLKQLRKSGKVPAVVYGYGTKNVSVKVDEVEFIKVIREVGRNGVIELGVGSKTIKVMVADYQFDPLKNQITHIDFLAINMSEERTVEVPVQLVGEAVGAKEGGVVEQPLFNLEVTATPDNIPEAIEVDITELNINDSLTVADVKVTGDFKIENDSAESVVTVVAPTEEPTEEEIEAMEGEQQTEEPEVVGESKEDEEKTEE

SEQ ID NO: 38 - protein, 253 aa, Staphylococcus aureus
MRTPIIAGNWKMNKTVQEAKDFVNALPTLPDSKEVESVICAPAIQLDALTTAVKEGKAQGLEIGAQNTYFEDNGAFTGETSPVALADLGVKYVVIGHSERRELFHETDEEINKKAHAIFKHGMTPIICVGETDEERESGKANDVVGEQVKKAVAGLSEDQLKSVVIAYEPIWAIGTGKSSTSEDANEMCAFVRQTIADLSSKEVSEATRIQYGGSVKPNNIKEYMAQTDIDGALVGGASLKVEDFVQLLEGAK

SEQ ID NO: 39 - protein, 353 aa, Staphylococcus aureus
MSRITQVHRILEQKHLDAIIILSDYNRRYLSGFTGTSGALIISKDKQYLITDFRYIDQATKQAPNYEIINRKSTIIGEIKELLHQENFENIGFEGHHVSYDTYLELNKSRISLISISNTVDKIRDVKDVDEIALIQKAANIVDETYEYILTVVKAGMTEKELKAILESKMLELGADGPSFDTIVASGHRGALPHGVASDKIIEKGDMITLDFGAYYNGYCSDITRTFAIGEPDPKLKEIYQIVLESQMKAINEIRPGMTGAEADAISRNYLESKGYGKEFGHSLGHGIGLEIHEGPMLARTIQDKLQVNNCVTVEPGVYIEGLGGIRIEDDILITENGCQVFTKCTKDLIVLT

SEQ ID NO: 40 - DNA, 23 bp, Synthetic
caccatgccaattattacagatg

SEQ ID NO: 41 - DNA, 28 bp, Synthetic
ttatttatctaagttatagaatgatttg

SEQ ID NO: 42 - DNA, 25 bp, Synthetic
caccatgaaaatgactaagagtgct

SEQ ID NO: 43 - DNA, 26 bp, Synthetic
tacatgtacattccaccatttacatg

SEQ ID NO: 44 - DNA, 25 bp, Synthetic
caccttgattagaaaccgtgttatg

SEQ ID NO: 45 - DNA, 27 bp, Synthetic
ttagttattttgtgttacatcctcatc

SEQ ID NO: 46 - DNA, 921 bp, Staphylococcus aureus
ttgattagaaaccgtgttatgaatagcgttgtcaataaatatttgcttcacaatcgctcgattatgtttaaaaatgatcaagatgttgaaagatttttttataaaagagaaattgaaaatagaaaaaaacataagcagccttcaacattaaatgttaaagcaaatttagaaaaattatcattagatgatatgcaagtctttcgctttaatttcagacatcaaattgataagaaaattttatatattcacggtggattcaatgcactacaaccatcaccgttccattggagattgttggataaaatcactttaagtacattatatgaggttgtactgcctatctatccaaagacaccagagtttcatattgacgatactttccaagcgatacaacgtgtttatgatcaattagtatctgaagtaggacatcaaaatgtcgtagtcatgggtgatggttcaggtggtgcactggcattatcctttgtacaatctcttttagataatcaacagccattaccgaataaattgtatttaatctcaccaattttagatgcaacactatctaataaagatatttcggacgccttaatcgaacaagatgcggttctaagtcagtttggtgtcaatgagattatgaaaaaatgggcgaatggcctaccattaacagataagcgcatatcgccaattaatggcacaatagaaggattgccaccagtttatatgtttggtggtggacgtgaaatgacacatcctgatatgaagctattcgaacaaatgatgttgcaacatcatcaatacattgagttttatgattatcctaagatggtacatgattttccaatttatccaattagacaatcacataaagctattaaacaaattgccaaatcgatagatgaggatgtaacacaaaataactaa

SEQ ID NO: 47 - DNA, 1305 bp, Staphylococcus aureus
atgccaattattacagatgtttacgctcgcgaagtcttagactctcgtggtaacccaactgttgaagtagaagtattaactgaaagtggcgcatttggtcgtgcattagtaccatcaggtgcttcaactggtgaacacgaagctgttgaattacgtgatggagacaaatcacgttatttaggtaaaggtgttactaaagcagttgaaaacgttaatgaaatcatcgcaccagaaattattgaaggtgaattttcagtattagatcaagtatctattgataaaatgatgatcgcattagacggtactccaaacaaaggtaaattaggtgcaaatgctattttaggtgtatctatcgcagtagcacgtgcagcagctgacttattaggtcaaccactttacaaatatttaggtggatttaatggtaagcagttaccagtaccaatgatgaacatcgttaatggtggttctcactcagatgctccaattgcattccaagaattcatgattttacctgtaggtgctacaacgttcaaagaatcattacgttggggtactgaaattttccacaacttaaaatcaattttaagcaaacgtggtttagaaactgcagtaggtgacgaaggtggtttcgctcctaaatttgaaggtactgaagatgctgttgaaacaattatccaagcaatcgaagcagctggttacaaaccaggtgaagaagtattcttaggatttgactgtgcatcatcagaattctatgaaaatggtgtatatgactacagtaagttcgaaggcgaacacggtgcaaaacgtacagctgcagaacaagttgactacttagaacaattagtagacaaatatcctatcattacaattgaagacggtatggacgaaaacgactgggatggttggaaacaacttacagaacgtatcggtgaccgtgtacaattagtaggtgacgatttattcgtaacaaacactgaaattttagcaaaaggtattgaaaacggaattggtaactcaatcttaattaaagttaaccaaatcggtacattaactgaaacatttgatgcaatcgaaatggctcaaaaagctggttacacagcagtagtttctcaccgttcaggtgaaacagaagatacaacaattgctgatattgctgttgctacaaacgctggtcaaattaaaactggttcattatcacgtactgaccgtattgctaaatacaatcaattattacgtatcgaagatgaattatttgaaactgctaaatatgacggtatcaaatcattctataacttagataaataa

SEQ ID NO: 48 - DNA, 741 bp, Staphylococcus aureus
atgaaaatgactaagagtgctttagtaacaggtgcatcaagaggaattggacgtagtattgcgttacaattagcagaagaaggatataatgtagcagtaaactatgcaggcagcaaagagaaagctgaagcagtagtcgaagaaatcaaagctaaaggtgttgacagttttgcgattcaagcaaatgttgccgatgctgatgaagttaaagcaatgattaaagaagtagttagccaatttggttctttagatgttttagtaaataatgcaggtattactcgcgataatttattaatgcgtatgaaagaacaagagtgggatgatgttattgacacaaacttaaaaggtgtatttaactgtatccaaaaagcaacaccacaaatgttaagacaacgtagtggtgctatcatcaatttatcaagtgttgttggagcagtaggtaatccgggacaagcaaactatgttgcaacaaaagcaggtgttattggtttaactaaatctgcggcgcgtgaattagcatctcgtggtatcactgtaaatgcagttgcacctggttttattgtttctgatatgacagatgctttaagtgatgagcttaaagaacaaatgttgactcaaattccgttagcacgttttggtcaagacacagatattgctaatacagtagcgttcttagcatcagacaaagcaaaatatattacaggtcaaacaatccatgtaaatggtggaatgtacatgtaa

SEQ ID NO: 49 - DNA, 1209 bp, Staphylococcus aureus
atggctcaagatcgtaaaaaagtacttgtacttggtgctggttatgcaggtttacaaactgtaactaaattgcaaaaagcgatatcaacagaagaagcagaaattacgcttattaataaaaatgaatatcactatgaagcaacatggttacatgaagcatcagcaggtacactaaactatgaagatgtattatatcctgtggaaagtgtcttgaagaaagacaaagtgaactttgttcaagcagaagtaacaaaaattgaccgtgatgctaaaaaggtagaaacaaatcaaggtatttatgactttgatattttagtagtagcattaggtttcgttagtgaaacattcggcatcgaaggtatgaaagatcatgctttccaaattgaaaatgttatcacagcacgtgaattatcacgtcatatcgaagacaaatttgctaactatgcagcatcaaaagaaaaagatgataacgatttatctatcttagttggtggtgctggattcactggtgttgaattcttaggtgaattaacagacagaattcctgaattatgtagcaaatatggtgtggatcaaaataaagttaaaatcacttgtgttgaagcagcacctaaaatgttaccaatgttctcagaagaattagttaaccacgcagttagctacttagaagaccgcggtgttgaatttaaaattgctacaccaatcgttgcttgtaacgaaaaaggttttgtagttgaagtagatggtgaaaaacaacaattaaatgcaggtacttcagtatgggcagctggtgtacgtggtagtaaattaatggaagaatcatttgaaggcgttaaacgtggacgtatcgttacaaagcaagatttaacaatcaatggttacgacaacatttttgttattggtgactgttcagcgtttatcccagctggagaagaacgtccattaccaactacagcacaaattgcaatgcaacaaggtgaaagtgttgctaaaaacattaaacgcatcttaaacggtgaatcaactgaagaattcgaatatgttgatcgtggaactgtttgttctttaggttcacatgacggtgtaggtatggtatttggtaaacctatcgctggtaaaaaagcagcattcatgaaaaaagtgattgatacacgtgcggtattcaaaatcggtggtatcggtttagcattcaaaaaaggtaaattctag

SEQ ID NO: 50 - DNA, 1941 bp, Staphylococcus aureus
atgagttcacaaaaaaagaaaattagtctttttgcgttcttcttattaaccgtaataacgattaccttgaagacgtatttttcttattatgttgatttttctttaggtgttaaaggtttagtacaaaacttaatattattgatgaatccttatagtttagtagcactggttttaagtgtgttcctattctttaaaggcaaaaaagcattttggttcatgttcataggcggcttcttattgacgttcctattatatgccaatgttgtgtactttagattcttctctgattttttaacgtttagtactttaaaccaagtaggtaacgtagaatctatgggtggtgcggttagtgcatcattcaaatggtatgactttgtttatttcattgatacgttagtttacttattcattttaatatttaaaacaaaatggttagacacaaaagcatttagtaagaaatttgttcctgtcgtaatggcagcttcagtagcattattcttcttaaacttagcttttgctgaaactgacagaccagaattattaacacgtacatttgaccataaatatttagtgaaatatttaggaccttataactttacagtatacgatggtgttaaaactatcgaaaataatcaacaaaaagcgttagcatctgaagatgacttaacaaaagtattaaattatacgaaacaacgtcaaacagagcctaacccagaatattatggggtggcaaagaagaaaaatattattaagattcatttagaaagtttccaaaccttcttaattaataaaaaggttaatggtaaagaagtaacaccgtttttaaacaaattatcaagtgggaaagagcaattcacatacttccctaactttttccatcaaacaggtcaaggtaaaacatctgactctgaatttacaatggataacagtttatacggtttaccgcaaggttctgccttttcattaaaaggagataatacgtatcagtcattaccagcaattttagatcaaaagcaaggctacaaatctgatgtcatgcacggtgactataaaacattctggaacagagaccaagtatataaacactttggtatcgataaattctatgatgcaacatactatgacatgtcagataaaaacgttgtaaacttaggcttgaaagacaaaattttctttaaagattctgctaattatcaagctaagatgaaatcaccattctattctcatttaattacattgactaaccactatccattcacattagatgaaaaggatgcgactattgagaagtcaaacacaggtgatgcaacagttgatggttatattcaaacagcgcgttatttagacgaagcattagaagaatatattaatgacttgaagaaaaaaggattatatgacaattcagtgattatgatttatggtgaccactatggtatctctgaaaaccataacaatgccatggaaaaactattaggtgaaaaaatcacaccagctaaatttacagatttaaacagaactggtttctggattaaaatccctggtaaatctggtggtatcaataatgaatatgctggtcaagtcgatgtaatgccaacaattttacatttggctggtatagatacgaagaattatttaatgttcggtactgatttattctctaaaggtcataatcaagtagttccattcagaaatggtgactttataacaaaagattataaatatgttaatggtaagatttattctaataaaaataatgaactcataactactcaaccagctgatttcgaaaagaataaaaagcaagttgaaaaggatctcgaaatgagtgacaacgtgcttaatggtgatttgtttagattctacaaaaatccagacttcaaaaaggtaaatccttcgaagtataaatatgaaacaggacctaaagcaaactctaaaaaataa

SEQ ID NO: 51 - DNA, 969 bp, Staphylococcus aureus
atggaggatgttttatacatgaaaaaattaacagcagcagcgattgcaacgatgggcttcgctacatttacaatggcgcatcaagcagatgcagcagaaacgacaaacacccaacaagcacatacacaaatgtcaacacaatcacaagacgtatcttatggtacttattatacaattgattctaatggggattatcatcacacacctgatggtaactggaatcaagcaatgtttgataataaagaatatagctatacattcgtagatgctcaaggacatacgcattatttttataactgttatccaaaaaatgcaaatgccaatggaagcggccaaacatatgtgaatccagcaacagcaggagataacaatgactacacagcgagtcaaagccaacagcatattaatcaatatggttatcaatcaaatgtaggtccagacgcgagctattattcacatagtaacaacaaccaagcgtataacagccatgatggtaatggaaaggtcaattatcctaatggcacatctaatcaaaatggtggatcagcaagtaaagcgacagctagtggtcatgcgaaagacgcaagctggttaacaagtcgtaaacaactacaaccatatggacaatatcacggtggtggtgcgcattacggtgtcgactatgcaatgcctgaaaattcaccagtttactcattaactgatggtacagtagtacaagcaggttggagtaactatggtggcggcaatcaagtaacgattaaagaagcgaacagtaataactaccaatggtatatgcataataatcgtttaactgtttcagctggtgataaagtcaaagctggtgaccaaattgcatattcaggtagtacgggtaattcaacagcgcctcacgtacacttccaacgtatgtctggtggcatcggtaatcaatatgcagtagacccaacgtcatacttgcaaagtagataa

SEQ ID NO: 52 - DNA, 825 bp, Staphylococcus aureus
atgcaaccacatttaatatgtctagacttagacggaacattattaaacgataacaaagaaatttcatcatatactaaacaagtattaaatgaattacaacaacgtggacaccaaattatgattgcgactggcagaccttatcgtgcaagtcaaatgtattatcatgaattaaatttaacgacaccaattgttaattttaatggcgcttacgtacatcaccctaaagataaaaacttcaaaacttgccatgaaattttagatttaggcatcgcacaaaacattattcaaggattacaacaatatcaagtatcgaatattatagcagaagtgaaagattatgttttcattaacaatcatgatccaagattatttgaaggtttttcaatgggtaatccaagaattcaaactggtaatttacttgtccacttgaaagaatcccctacctcaattttaattgaagccgaagaaagtaaaatacctgaaatcaaaaatatgcttactcatttttatgccgatcatattgagcatcgacgctggggcgcaccattccctgtcattgaaattgtaaaacttggtattaataaagcaagaggcattgagcaagttagacaatttttaaatattgaccgaaataatattattgcattcggtgatgaagataatgatattgaaatgattgagtacgcccgccatggtgttgctatggaaaatggtttgcaagaacttaaagatgtagcgaacaatattacattcaacaataatgaagatggcattggtcgatatttgaatgatttctttaatttaaatattagatattactgttaa

SEQ ID NO: 53 - DNA, 702 bp, Staphylococcus aureus
atgaaaaagacaattatggcatcatcattagcagtggcattaggtgtaacaggttacgcagcaggtacaggacatcaagcacacgctgctgaagtaaacgttgatcaagcacacttagttgacttagcgcataatcaccaagatcaattaaatgcagctccaatcaaagatggtgcatatgacatccactttgtaaaagatggtttccaatataactttacttcaaatggtactacatggtcatggagctatgaagcagctaatggtcaaactgctggtttctcaaacgttgcaggtgcagactacactacttcatacaaccaaggttcagatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgtttcagcttctacatgggctgcaatcatcgctcgtgaatcaaatggtcaagtaaatgcttacaacccatcaggtgcttcaggtttattccaaactatgccaggttggggtccgacaaacactgttgaccaacaaatcaacgcagctgttaaagcatacaaagcacaaggtttaggtgcttggggattctaa

SEQ ID NO: 54 - DNA, 1380 bp, Staphylococcus aureus
atgaatatcattgagcaaaaattttatgacagtaaagcttttttcaatacacaacaaactaaagatattagttttagaaaagatcaattaaagaagttaagcaaagctattaaatcatacgagagcgatattttagaagcactatatacagatttaggaaaaaataaagtcgaagcttatgctactgaaattggcataactttgaaaagtatcaaaaatgcccgtaaggaacttaaaaactggactaaaacaaaaaatgtagacacacctttatatttatttccaacaaaaagctatatcaaaaaagaaccttatggaacagttttgatcattgcaccatttaactatccttttcaactagtattcgaacctttaatcggtgctattgcagcaggtaatacagcaattattaaaccatctgagttgacaccaaatgttgcacgagtgattaaacgattaatcaatgaaacatttgatgcaaattacattgaagttattgagggaggaattgaagaaacgcaaacgttaattcacttaccttttgactatgtcttctttacaggaagtgaaaatgtaggcaaaatcgtttatcaagctgccagcgaaaatttagttcctgtgacattagaaatgggtggtaaatctccagtcatcgttgatgaaacagcgaatattaaagttgctagtgagcgcatttgttttgggaaattcactaatgccggccaaacatgtgttgcaccagattacattttagtacacgaatctgtaaaagatgatttaatcacagccctatcaaaaacgttgcgtgaattttatggtcaaaatatacaacaaagtccagattatggccgcattgtaaaccttaaacattatcatcgtctgacttcattacttaacagtgcacaaatgaatattgtatttggtggtcatagtgatgaggatgaacgttatatagaaccaacattgttagatcacgttacaagtgattcagcaattatgcaagaagaaatttttggtcctatcttaccgattttaacgtatcagtcattggatgaagcaatagcctttattcaccaaagaccaaaacctttgagtttatatttatttagcgaagatgaaaatgctacacaacgtgtaataaacgagttatcatttggcggcggcgctattaatgatacattgatgcacctagcgaatcctaaattaccatttggtggtgttggcgcctcaggtatgggacgctatcatggtaaatattcattcgacacttttacacatgaaaaaagctacattttcaaatctacacgattagaatcaggtgtccatttaccaccatataaaggtaaatttaaatacatcaaagctttctttaaaaattaa

SEQ ID NO: 55 - DNA, 870 bp, Staphylococcus aureus
attagggactccaaacccaataaatactgttgttacaaggtttctatgtatccaaactggggacaatataaacgcgctgatttaatcggacaatcttcttatattaaaaataatgatgtcgtaatattcaatgaagcatttgataatggtgcatcagacaaattattaagtaatgtgaaaaaagaatatccttaccaaacacctgtactcggtcgttctcaatcaggttgggacaaaactgaaggtagctactcatcaactgttgctgaagatggtggcgtagcgattgtaagtaaatatcctattaaagaaaaaatccagcatgttttcaaaagcggttgtggattcgataatgatagcaacaaaggctttgtttatacaaaaatagagaaaaatggtaagaacgttcacgttatcggtacacatacacaatctgaagattcacgttgtggtgctggacatgatcgaaaaattagagctgaacaaatgaaagaaatcagtgactttgttaaaaagaaaaatatccctaaagatgaaacggtatatataggtggcgaccttaatgttaataaaggcactccagagttcaaagatatgcttaaaaacttgaatgtaaatgatgttctatatgcaggtcataatagcacatgggaccctcaatcaaattcaattgcgaaatataattaccctaatggtaaaccagaacatttagactatatatttacagataaagatcataaacaaccaaaacaattagtcaatgaagttgtgactgaaaaacctaagccatgggatgtatatgcgttcccatattactacgtttacaatgatttttcagatcattacccaatcaaagcctatagtaaatag

SEQ ID NO: 56 - DNA, 804 bp, Staphylococcus aureus
atgaagaaaatcgctacagctactatcgcaactgcaggattcgctacaatcgcaattgcatcaggaaatcaagctcatgcttctgagcaagataactacggttataatccaaacgacccaacatcatatagctatacttacactattgatgcacaaggtaactaccattacacatggaaaggtaactggcatccaagtcaattaaaccaagataatggctactacagctattactactacaatggctacaataactacaacaattacaacaatggttatagctacaataattacagccgttacaacaactactcaaataataatcaatcatataactacaataactataatagttacaacacaaacagctaccgtactggtggtttaggtgcaagctacagcacttcaagcaacaatgttcaagtaactacaactatggctccatcatcaaatggccgttcaatctcaagtggttatacttcaggacgtaacttatacacttctggtcaatgtacatactacgtatttgatcgtgtaggtggtaaaatcggttcaacttggggcaatgcaagtaactgggctaacgcagctgcaagagctggttacacagtgaacaatacaccaaaagctggtgcaattatgcaaacaactcaaggtgcatacggtcacgttgcatacgttgaaagtgttaacagcaatggttcagtaagagtttcagaaatgaactatggttatggcccaggtgttgtaacttcacgtacaatctcagctagccaagctgctggttataacttcattcactaa

SEQ ID NO: 57 - DNA, 438 bp, Staphylococcus aureus
atgcgtcaaacatttatggcaaatgaatcaaacattgagcgcaaatggtatgttatcgatgctgaaggccaaacattaggtcgtttatcatcagaagtagcatctatcttacgcggtaaaaataaagtaacttacacaccacacgttgatactggtgattatgtaatcgttattaatgcatcaaaaatcgaatttactggtaacaaagaaactgacaaagtttactaccgtcactcaaatcatccaggtggtatcaaatcaatcactgctggtgaattaagaagaactaacccagaacgtttaattgaaaactcaattaaaggtatgttaccaagcactcgtttaggcgaaaaacaaggtaaaaaattatttgtatatggtggcgctgaacatccacacgctgcacaacaaccagaaaactacgaattacgtggttaa

SEQ ID NO: 58 - DNA, 918 bp, Staphylococcus aureus
atggagacttatgaatttaacattacagataaagaacaaacaggtatgcgtgtagataagttgctgcctgaattaaataatgattggtctcgtaaccagatacaagattggattaaagcaggtttagtcgttgcaaacgataaagttgttaaatctaattataaagtgaaacttaatgatcatatagttgtcactgaaaaagaagtggttgaagctgatattctacctgaaaatttaaatttagatatttattatgaagatgacgatgttgcagttgtatataaaccgaaaggcatggtagttcatccatcaccagggcattataccaatacattagttaatggtttaatgtatcaaattaaaaatttgtcaggtattaatggagaaattcgtccaggtattgttcaccgtatagatatggatacttctggtttattaatggttgctaaaaatgatattgctcatcgtgggcttgtagaacaattaatggataaatctgttaaaagaaaatatatcgctttagttcacgggaatattcctcatgattacggtacaatcgatgcgccaattggtagaaacaaaaatgatcgtcaatctatggctgttgttgatgatggtaaggaagcagtgacacattttaacgtactagaacattttaaagattatacgcttgttgaatgtcaacttgaaacaggacgtacgcatcaaatccgtgtgcacatgaaatatattggcttcccattagttggtgatccaaagtatggaccgaaaaagacattggatattggtggtcaagctctacatgctggacttattggattcgaacatccagtaacaggtgaatatattgaaagacatgctgaattaccacaagactttgaagatttattagatacaattcgaaaaagagatgcataa

SEQ ID NO: 59 - DNA, 1185 bp, Staphylococcus aureus
atggcaaaagaaaaattcgatcgttctaaagaacatgccaatatcggtactatcggtcacgttgaccatggtaaaacaacattaacagcagcaatcgctactgtattagcaaaaaatggtgactcagttgcacaatcatatgacatgattgacaacgctccagaagaaaaagaacgtggtatcacaatcaatacttctcacattgagtaccaaactgacaaacgtcactacgctcacgttgactgcccaggacacgctgactacgttaaaaacatgatcactggtgctgctcaaatggacggcggtatcttagtagtatctgctgctgacggtccaatgccacaaactcgtgaacacattcttttatcacgtaacgttggtgtaccagcattagtagtattcttaaacaaagttgacatggttgacgatgaagaattattagaattagtagaaatggaagttcgtgacttattaagcgaatatgacttcccaggtgacgatgtacctgtaatcgctggttcagcattaaaagctttagaaggcgatgctcaatacgaagaaaaaatcttagaattaatggaagctgtagatacttacattccaactccagaacgtgattctgacaaaccattcatgatgccagttgaggacgtattctcaatcactggtcgtggtactgttgctacaggccgtgttgaacgtggtcaaatcaaagttggtgaagaagttgaaatcatcggtttacatgacacatctaaaacaactgttacaggtgttgaaatgttccgtaaattattagactacgctgaagctggtgacaacattggtgcattattacgtggtgttgctcgtgaagacgtacaacgtggtcaagtattagctgctcctggttcaattacaccacatactgaatttaaagcagaagtatacgtattatcaaaagacgaaggtggacgtcacactccattcttctcaaactatcgtccacaattctatttccgtactactgacgtaactggtgttgttcacttaccagaaggtactgaaatggtaatgcctggtgataacgttgaaatgacagtagaattaatcgctccaatcgcgattgaagacggtactcgtttctcaatccgcgaaggtggacgtactgtaggatcaggcgttgttactgaaatcattaaataa

SEQ ID NO: 60 - DNA, 2832 bp, Staphylococcus aureus
atgttaggagtaataaatagaatggcgaaaaaattcaattacaaactaccatcaatggttgcattaacgcttgtaggttcagcagtcactgcacatcaagttcaagcagctgagacgacacaagatcaaactactaataaaaatgttttagatagtaataaagttaaagcaactactgaacaagcaaaagctgaggtaaaaaatccaacgcaaaacatttctggcactcaagtatatcaagaccctgctattgtccaaccaaaaacagcaaataacaaaacaggcaatgctcaagtaagtcaaaaagttgatactgcacaagtaaatggtgacactcgtgctaatcaatcagcgactacaaataatacgcagcctgttgcaaagtcaacaagcactacagcacctaaaactaacactaatgttacaaatgctggttatagtttagttgatgatgaagatgataattcagaacatcaaattaatccagaattaattaaatcagctgctaaacctgcagctcttgaaacgcaatataaagccgcagcacctaaagctaaaactgaagcgacacctaaagtaactacttttagcgcttcagcacaaccaagatcagttgctgcaacaccaaaaacgagtttgccaaaatataaaccacaagtaaactcttcaattaacgattacattcgtaaaaataacttaaaagcacctaaaattgaagaagattatacatcttacttccctaaatacgcataccgtaacggcgtaggtcgtcctgaaggtatcgtagttcatgatacagctaatgatcgttcgacgataaatggtgaaattagttatatgaaaaataactatcaaaacgcattcgtacatgcatttgttgatggggatcgtataatcgaaacagcaccaacggattacttatcttggggtgtcggtgcagtcggtaaccctagattcatcaatgttgaaatcgtacacacacacgactatgcttcatttgcacgttcaatgaataactatgctgactatgcagctacacaattacaatattatggtttaaaaccagacagtgctgagtatgatggaaatggtacagtatggactcactacgctgtaagtaaatatttaggtggtacggaccatgccgatccacatggatatttaagaagtcataattatagttatgatcaattatatgacttaattaatgaaaaatatttaataaaaatgggtaaagtggcgccatggggtacgcaatttacaactacccctactacaccatcaaaaccaacaacaccgtcgaaaccatcaactggtaaattaacagttgcagcaaacaatggtgtcgcacaaatcaaaccaacaaatagtggtttatatactactgtttacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaatctggtacgaaactttatacagtaccttggggtacatctaaacaagttgctggtagcgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaatacctaagccatcaacacctacaacaaataataaattaacagtttcatcattaaacggtgttgctcaaattaatgctaaaaacaatggcttattcactacagtttatgacaaaactggtaagccaacgaaagaagttcaaaaaacatttgctgtaacaaaagaagcaagtttaggtggaaacaaattctacttagttaaagattacaatagtccaactttaattggttgggttaaacaaggtgacgttatttataacaatgcaaaatcacctgtaaatgtaatgcaaacatatacagtaaaaccaggcactaaattatattcagtaccttggggcacttataaacaagaagctggtgcagtttctggtacaggtaaccaaacttttaaagcgactaagcaacaacaaattgataaatctatctatttatttggaactgtaaatggtaaatctggttgggtaagtaaagcatatttagctgtacctgctgcacctaaaaaagcagtagcacaaccaaaaacagctgtaaaagcttatactgttactaaaccacaaacgactcaaacagttagcaagattgctcaagttaaaccaaacaacactggtattcgtgcttctgtttatgaaaaaacagcgaaaaacggtgcgaaatatgcagaccgtacgttctatgtaacaaaagagcgtgctcatggtaatgaaacgtatgtattattaaacaatacaagccataacatcccattaggttggttcaatgtaaaagacttaaatgttcaaaacctaggcaaagaagttaaaacgactcaaaaatatactgttaataaatcaaataacggcttatcaatggttccttggggtactaaaaaccaagtcattttaacaggcaataacattgctcaaggtacatttaatgcaacgaaacaagtatctgtaggcaaagatgtttattatacggtactattaataaccgcactggttggtaaagcaaaagatttaccgcaccaactcggaaaccaactacatcagctgccaaagatttaa

SEQ ID NO: 61 - DNA, 1695 bp, Staphylococcus aureus
atgactgattatgttactaaatacgcaaaaaaggtagtttcaggagaaattttggcaagtttgaagaatattcaagtatgcaaacgtcacctatcttttatggagaacccgccgaatggttgccattgggataatcatttgtctaacaaagcaattaaatttgtggaaatgcttccagaccctaaaacaaaccagcccatgcctcttatggagtttcagaaattcattgttgggagcttatacggctggcgtagaggtcaatacagaatgtttactaaagcttatataagtatggctagaaagcaaggtaagtctctaatcgtatcgggaatgtccgttaacgaactgttgtttggacaataccctaaatttaatagacaaatttatgtagcttcatctacttataagcaagcgcaaacaatattcaagatggcaagccaacaagtaaacctaatgcgaagtaaaagcaagtttatccgtgaaaaaacagacgtaagaaagacagacattgaagatgtattaagtagttcagtgtttgcacctctttccaataacccagatgcggttgatggtaaagatcctacagttgctattttggacgaattggcaagtatgcctgatgatgagatgtactcaaggtttaaaacaggtatgacattacaaaaaaatcctttaaccctacttgtttcaacggccggagacaatttaaatagtcaaatgtaccaagagtataagtatattaaacgtattttaaatgaagaagtaagagctgataattactttgtatattgtgctgaaatggattcacaagaagaagttcaagatgaaacaaagtggattaaagcaatgccgcttttagaatcaaaagaacatagaaaaactatacttcaaaatgtaaaagctgatatacaagacgaattagaaaaagggacatcgtatcataagattttgattaaaaacttcaatttatggcaagcgcaaagagaagatagcttgctagatatttcagattgggaacaagtaataacgcctatgcctaatatcaatggtaaagatgtgtatataggtgtcgacttatcgagattggatgacttaacatctgtagggtttattttccctaacgacgataaaaaagtgtttttacatagtcattctttcattggattaagaacaaacttagaacaaaaatctaagagagacaaaataaattatgaattagcgattgaacgtggagaagctgagactacacaatcagatagcggcatgattgattataaacaagttatcgattttatagtgaaatttataacgacgcatgacctgaatgtacaggctgtttgctatgacccttggaatgcgcaaagttttataacaacaatcgaatcaatggctttagattggccactcattgaagtgggacaaagttttaaggcgttatcacaatctattaaagaatttagaatgtgggttgcagatgaaagaatacagcataacgataatatgttacttacaacatcagttaataatgccgttttgattcgtgacggagaagacaatgtgaaaataaataaaaaaatgaatcgtcaaaaaatagatccgattatttcgattatcacagctttcactgaagctagaatgcacgaattccaagaaaattggacggagaaatatgaaagcgaagaattcggattttaa

SEQ ID NO: 62 - DNA, 1401 bp, Staphylococcus aureus
gtgactaaatcagtggctattataggagcggggataacaggtttatcaagtgcatattttttaaaacagcaagatcctaatattgatgtaaccatctttgaagcatcgaatcgtccggggggaaagattcaatcgtatcgtaaagatggttatatgattgaactagggcctgaatcttatttaggtagaaaaacgattatgacagaattagcgaaagatattggattagaacaagatattgttacaaatacgactggacaatcatatatttttgcgaaaaataaattatatccgattccaggtggttcaattatgggtattccaacagatattaaaccatttgttactacaaaattaatatcgccacttggtaaattaagagcaggattagatttaatcaaaaagcctatacaaatgcaagatggtgacatttctgttggtgcatttttcagagcaagattaggtaatgaggtacttgagaacttaattgagcctttaatgggtggtatttatggtaccgatattgataaattaagtttgatgagtacgtttcctaattttaaagaaaaagaagaggcattcggaagtctgataaaaggtatgaaggatgagaaaaataagcgtctgaaacaaagacaattatatcctggcgcaccaaaaggacaattcaaacaatttaagcatggtttaagctcatttattgaagcattagaacaagatgtgaaaaataaaggtgtgacaatacgctacaatacgtcagtggatgatattattacatctcaaaagcaatataaaattgtttacagtaatcaacaagaagatgtattcgatggggtattagtgacaacaccgcatcaagtctttttgaattggttcggacaagatccagcatttgattactttaaaacgatggatagtacgactgttgcaactgttgtattggcatttgatgaaaaagacattgaaaatacttatgatggtactggcttcgtgattgcgagaacgagtgatacagacattaccgcatgtacttggacatcgaaaaaatggccatttactacaccagaaggtaaggttttgattcgtgcgtatgtaggtaaaccaggtgatactgtggttgatgatcatacagataatgaattagtatcgattgtacgtagagatttaagtcaaatgatgacatttaaaggtgatcctgaatttacaattgtcaatcgtttgccgaaaagtatgccacagtaccatgtcggtcatattcaacaaattagacagattcaagcacatattaaacaaacatatccacgacttagagtaactggtgcatcttttgaagcggttggactacctgattgtattacacaaggtaaagttgctgctgaagaagtaatcgcagagttgtaa

SEQ ID NO: 63 - DNA, 1017 bp, Staphylococcus aureus
atgattaaacaattatacaaaaacatcacaatttgtagtttagcaatatctactgcattaactgtatttccggcaacttcttatgcaaaaattaattctgaaattaaagctgtttctgagaagaatcttgatggtgatactaaaatgtatacacgtacagctacaacaagtgatagtcaaaaaaatattactcaaagcttacaatttaatttcttaactgaacctaattatgataaagaaacagtatttattaaagcaaaaggtacaattggtagtggtttgagaattttagacccaaatggttattggaatagtacattaagatggcctggatcttattcagtttcaattcaaaatgttgatgacaacaacaatacaaatgtgactgactttgcaccaaaaaatcaggatgaatcaagagaagttaaatatacgtatggttataaaacaggtggagatttttcgattaatcgtggaggcttaactggaaatattacaaaagagagtaattattcagagacgattagttatcaacaaccatcatatcgtacattacttgatcaatctacgtcacataaaggtgtaggttggaaagtagaagcacatttgataaataatatgggacatgaccatacgagacaattaactaatgatagtgataatagaactaaaagtgaaatcttttctttaacacgaaatggaaatttatgggcgaaagataatttcacacctaaagacaaaatgcctgtaactgtgtctgaagggtttaatccagaatttttagctgttatgtcacatgataaaaaagacaaaggtaaatcacaatttgttgttcattataaaagatcaatggatgagtttaaaatagattggaatcgccatggtttctggggctattggtctggtgaaaaccatgtagataaaaaagaagaaaaattatcagcattatatgaagttgattggaagacacatgatgtgaagtttgtaaaagtacttaatgataatgaaaagaaataa

SEQ ID NO: 64 - DNA, 1458 bp, Staphylococcus aureus
atgagcattcgctacgaatcggttgagaatttattaactttaattaaagacaaaaaaatcaaaccatctgatgttgttaaagatatatatgatgcaattgaagagactgatccaacaattaagtcttttctagcgctggataaagaaaatgcaattaaaaaagcgcaagaattggatgaattacaagcaaaagaccaaatggatggcaaattatttggtattccaatgggtataaaagataacattattacaaacggattagaaacaacatgtgcaagtaaaatgttagaaggttttgtgccaatttacgaatctactgtaatggaaaaactacataatgagaatgccgttttaatcggtaaattaaatatggatgagtttgcaatgggtggttcaacagaaacatctatttcaaaaaaaacagttaacccatttgaccataaagcagtaccaggtggttcatcaggtggatctgcagcagcagttgcagctggcttagtaccatttagcttaggttcagacactggtggttcaattagacaaccggctgcatattgtggtgttgtcggtatgaaaccaacatacggtcgtgtatctcgatttggattagttgcatttgcatcttcattagaccaaattggtccattgactcgaaatgtaaaagataatgcaatcgtattagaagctatttctggtgcagatgctaatgactctacaagtgcacctgttgatgatgtagactttacatctgaaattggtaaagatattaaaggattaaaagttgcattacctaaagaatacttaggtgaaggtgtagctgatgacgtaaaagaagcagttcaaaacgctgtagaaactttaaaatctttaggtgctgtcgttgaggaagtatcattgccaaatactaaatttggtattccatcatattacgtgattgcatcatcagaagcttcgtcaaacctttctcgttttgacggaattcgttatggttatcattctaaagaagctcattcattagaagaattatataaaatgtcaagatctgaaggtttcggtaaagaagtaaaacgtcgtattttcttaggtacatttgcattaagttcaggttactacgatgcttactataaaaaatctcaaaaagttagaacattgattaaaaatgactttgataaagtattcgaaaattatgatgtagtagttggtccaacagcgcctacaactgcgtttaatttaggtgaagaaattgatgatccattaacaatgtatgccaatgatttattaacaacaccagtaaacttagctggattacctggtatttctgttccttgtggacaatcaaatggccgaccaatcggtttacagttcattggtaaaccattcgatgaaaaaacgttatatcgtgtcgcttatcaatatgaaacacaatacaatttacatgacgtttatgaaaaattataa

SEQ ID NO: 65 - DNA, 1242 bp, Staphylococcus aureus
gtggccgaacactcatttgacgttaatgaagtaatcaaggattttccgatattagatcaaaaagtcaatggcaaacgtttagcatatcttgattcaacagcgacaagtcaaacgcctatgcaagtgttaaatgttttagaggattactacaagcgttataattcaaacgttcatcgtggtgttcatacattaggatcattggcaactgatggttatgaaaatgcccgtgaaaccgttcgtcgttttattaatgcgaagtattttgaagaaatcattttcacacgcggaacaactgcgtcgattaaccttgtagcacatagctatggtgatgcaaatgttgaagagggcgatgaaattgttgtcactgaaatggagcatcatgccaatattgttccttggcaacagttagcaaagcgtaaaaatgcgacattgaaatttataccaatgacagctgacggtgaattaaacatcgaagatattaagcaaacgattaatgataaaacaaagatcgttgctattgcacatatttctaatgtactcggtacaattaatgatgttaaaaccattgcagaaatagctcatcaacatggcgcaattatcagtgttgatggggcgcaagcagcaccacatatgaaacttgatatgcaagaaatgaatgctgatttttatagttttagtggtcataaaatgcttggaccaacaggtattggcgtattatttggtaaacgtgagttactacaaaaaatggaaccgattgagttcggtggtgacatgattgattttgtaagtaagtatgatgcaacatgggctgatttacctactaaatttgaggcgggtactccattaattgctcaagcaattgggcttgcagaagctattcgctatttagaacgcataggttttgatgcaattcataaatatgaacaagaattaacgatatatgcttatgagcaaatgtctgcaattgaaggaattgaaatttatggcccgcctaaggatcgtcgtgcaggtgtaataacgtttaatttacaagatgtacatccacacgatgttgctacagccgtagatacagaaggtgtagcggttagagctgggcatcattgtgcgcaaccgttaatgaaatggttaaatgtgtcttcaacagctagagcgagtttttatatatacaacacgaaagaagacattgatcagttaataaatgccttgaaacaaacgaaggagtttttctcttatgaattttaa

SEQ ID NO: 66 - DNA, 1185 bp, Staphylococcus aureus; identical to SEQ ID NO: 59

SEQ ID NO: 67 - DNA, 780 bp, Staphylococcus aureus
atgcaagcattacaaacatttaattttgaagaattaccagtaagaacattagaggttgacggagaaccatattttataggaaaagatgttgctgacattttaggatatgcaaacggacgagatgctttgtcaaaacatgttgatgaagacgacaagaaagttctaacgtcgcgaaatacgactttagaaaatttaccaaatcgaggacttactgcagtcaacgaatcgggtttatacagcctaatcttctcatcaaaactagaatcagctaaacgattcaaacgctgggtaacatcagatgtcctaccagccattcgcaaatatggtatctacgcaacggacaacgtaattgaacaaacattaaaagatccagactacatcattacagtgttgactgagtataagaaagaaaaagagcaaaacttacttttgcaacaagaaatcggagagctaaaacccaaagcagattatgttgatgaaatcttaaaatcaactggcacattagctacaactcaaatcgcggcagactacggtatatcagcacaaaagttaaacaaactactacacgaagctagactacaacgaaaagtaaataaacagtgggtgctttactcagaacacatgggcaagagttacacagattcagacactataacaattgtgcgttctgatggcagagaagacacagttttacaaactagatggacacaaaaaggcagattgaaaatacatgaaatcatgactgaattcggttatgaagctaatttagggggagcgtaa

SEQ ID NO: 68 - DNA, 702 bp, Staphylococcus aureus
ttagaatccccaagcacctaaaccttgtgctttgtatgctttaacagctgcgttgatttgttggtcaacagtgtttgtcggaccccaacctggcatagtttggaataaacctgaagcacctgatgggttgtaagcatttacttgaccatttgattcacgagcgatgattgcagcccatgtagaagctgaaacaccagtacgttgagccatgatttgagctgctgatgaaccagtagcacctgcagtattaccattgcttaatctcactgaacttgaagtagttgaagtgctgtagttatggtaagttggagctgaaacagcttcaacgtttgagttacttgattgtgcattgtagcttactgattgtacatctgaaccttggttgtatgaagtagtgtagtctgcacctgcaacgtttgagaaaccagcagtttgaccattagctgcttcatagctccatgaccatgtagtaccatttgaagtaaagttatattggaaaccatcttttacaaagtggatgtcatatgcaccatctttgattggagctgcatttaattgatcttggtgattatgcgctaagtcaactaagtgtgcttgatcaacgtttacttcagcagcgtgtgcttgatgtcctgtacctgctgcgtaacctgttacacctaatgccactgctaatgatgatgccataattgtctttttcat

SEQ ID NO: 69 - DNA, 1413 bp, Staphylococcus aureus
ttatacttcaacacccatatctttagcttttgcaataacatcatccatgctaccaactaaacggaatgcatcttctggaatatggtcatatttaccatctaagatatctttaaagtttgcaactgttgtcttaacaggtacataagaacctttttgaccagtaaattgttccgctacgtggaagttttgagataagaagaattgaattctacgtgcgcgttcaactgtttgtttatcttcatcagataattcgtccatacctaagatagcaatgatatcttgcaattcacggtatttttgaagtgttgattgtacatcacgagctacttcataatgttcttgacctacaattgatggttccaatgctcttgatgtagacgctaatggatccacggctggataaatacccatttcagttaatttacgttctaagttagtagttgcatctaaatgggcaaacgctgtcgcaggcgctgggtcagtatagtcatcggcaggtacgaataccgcttgaatagaagtaactgatccttttgttgtagacgtaatacgttcttgtaattgtcccatttcagtagcaagtgttggttggtaacctactgcagaaggcatacgacctaataatgcagatacctcagaaccagcttgtgtaaatctgaaaatgttatcgatgaataataatacgtcttgaccttgttcgtcacggaaatattcagccattgttaaaccagataatgcaacacgcatacgtgcaccaggtggctcattcatttgcccgaataccatggctgttttcttaattacaccactgtcactcatttcgaagtataaatcgttaccttcacgagtacgttcacctacaccggcgaatacagaaataccaccgtgctcttgagcgatgttgttaattaattcttggattaatactgttttacctacaccggcaccaccgaacaatccgattttaccacctttaatataaggtgctagtaaatctactactttaatacctgtttctaaaatttgaacttctgttgaaagttcatcgaatgctggtgcttgacgatggataggatcgcggcgaacagaatcactaatttcttctttaaggtcaattgtttcacctagtacattaaatacacgacctaatgtttcgtcaccaacaggtacactaatttctttgcctgtatcttttacatccatgcctctttggacaccatcagttgaatccatcgcaattgtacgaacaacgtcgtcacctaattgcagcgcaacttctaatgttagttgtattgtaccttcttctttaggcacatcaataaccaaggcgttattaattttaggaacttcgttatgttcaaatcgaacatcaattacaggacccataacttgagttacacggccaattcccat

SEQ ID NO: 70 - DNA, 1119 bp, Staphylococcus aureus
ttataattgtaatgcttcttctacagatttatattccatttcaaatgcctctgcaacgcctttattggttacgtgacctttgtaagtatttaaacctaatgataatggttgatttgatttaaatgcttctctataccctttattagctagcatgagcgcataaggtagcgtagcattatttaaagctaacgtcgaagtacgcggtactgcacctggcatatttgcaactgcataatgaaccacaccatgcttaatatatgtaggatcatcatgtgtcgtaattttatcagttgtttcaaaaataccgccttgatcaatagcaatgtcaataataactgacccatttttcatttgtttaatcatgtcttctgttacaagtcttggcgctttagcacctggaattaaaactgcacctattactaaatcactttgtttaacatacaactcaatattcaacggatttgacataattgtatgtacacgtccaccgaataaatcatctaattgttgtaaacgctttggattaacatctaaaatcgtaacatctgcacctagtcctagtgcaattttagctgcatttgttcctgcttgaccaccaccgataatagttactttacccttaggtactcctgggacaccacctagtagaattcccataccaccattaagtttttgtaggaactctgcgccaacttgagctgacattcttcctgctacctcactcattggtgataacaatggtaaagatcggtctggtaactgcacagtctcatatgcaatactaattacttttctatctatcaaagcttgtgttaatttttcttcatttgctaaatgaagataagtgaataatacaagcccttctttaaaatatggatattcagattcaagtggttctttaactttaataaccatatccacatcccaaacttttgcttgttcagcaacaatctcagcacctgcttctttgtaatctacatcttcaaagaatgatcctgaacccgcatttgtttccactaaaacagtatgcccactttctactaaagcgtgcacaccacttggtgataaaccaacacgattttcattatttttaatctcccttggtataccaattttcat

SEQ ID NO: 71 - DNA, 1179 bp, Staphylococcus aureus
atgacaagaccatttaatcgtgtacatttaatcgtaatggattcagtaggtattggtgaagcgccagacgcagctgattttaaagatgaaggttcacatactttaagacataccttagaaggtttcgatcaaactttaccaaaccttgaaaagttaggtctagggaacatcgataaattaccagtagtaaatgcagttgaacaaccagaagcatactatactaaattgagtgaagcttcagttggtaaagatacaatgactggtcactgggaaattatgggattaaatattatgcaaccttttaaagtataccctaatggattccctgaagagttaattcaacaaattgaagaaatgacaggtcgtaaagttgttgctaacaaaccggcatcgggtacgcaaattatcgatgagtggggcgagcaccaaatgaaaactggtgacttaattgtttatacaagtgcagacccagtattgcaaattgctgcacatgaagacattatcccattagaagagttatatgatatttgtgaaaaggttcgtgagttgacaaaagaccctaaatatttaattggtcgtattatcgcacgtccatatgttggtgaaccaggaaactttacacgtacatctaatcgacatgactatgcgttaaaaccatttggtaaaactgtcttagatcatttgaaagacggtggttatgatgttattgccatcggtaaaattaatgacatttatgatggtgaaggtgtaacagaagcggttcgtacgaagagtaacatggacggtatggatcaattgatgaaaattgttaagaaagatttcacaggtattagcttcttaaacttagtagactttgatgcattatacggtcatcgtcgtgataaaccaggttatgcacaagcaattaaagatttcgatgatcgcttgccagaactgtttagcaacttaaaagaagacgatttagtaattattacagcagaccatggtaatgacccgacagcgccaggtacggaccatacgagagaatatatcccagtaattatgtacagtccgaaatttaaaggtggtcatgcactagaaagtgatactacattcagttctatcggtgcaactatagcagataatttcaacgtaacattaccagagttcggtaaaagttatttaaaggaattgaaatag

SEQ ID NO: 72 - DNA, 1245 bp, Staphylococcus aureus
atgactgagaacaataatttagtaacttctactcaaggaattattaaagaagcattgcataaattgggatttgacgaaggaatgtacgatttaattaaagaacctttaagaatgttacaagtgcgtatccctgtacgaatggatgatggcacagttaaaacattcacaggttaccgtgcgcaacataatgatgctgttggaccaacaaaagggggcgtgcgtttccacccagatgttgatgaagaagaagtaaaagcattatcaatgtggatgactttgaaatgtggcattgtaaacttaccatacggtggtggtaagggtggtatcgtttgtgatccacgtcaaatgagcattcatgaagttgaacgtttatcacgcggatatgtaagagcaatttcacaattcgtaggtccgaacaaagatattccagcaccagatgtatttacaaactcacaaattatggcttggatgatggatgaatatagtgcattagataaatttaattcaccaggtttcatcacaggtaaaccaattgtattgggtggttctcatggacgcgacagatcaactgcactaggtgtagttattgcaattgaacaagctgcaaaacgtcgtaatatgcaaattgaaggtgccaaggttgttattcaaggtttcggtaatgccggaagtttcttagctaaattcttatatgatttaggtgcaaaaattgtaggtatctctgatgcttacggtgcattacacgatccaaatggcttagatatagattatttattagaccgtcgtgatagttttggtacggtaacaaatttatttgaagaaacaatctcaaataaagaattgtttgaattagattgtgacattttagtaccagcggctatttcaaaccaaattacagaagacaatgcacatgatattaaagctagtatcgttgttgaagctgctaatggacctacaacaccagaagcaacacgtattttaactgaacgtggtatattattagttccagacgtattagcaagtgctggtggtgtaacggtttcttacttcgaatgggtacaaaataatcaaggttattattggtctgaagaagaagtaaatgaaaagctacgtgaaaaattagaagcggcatttgacacgatttacgaattgtctcaaaatcgaaaaatagatatgagacttgcagcatatatcataggtattaaacgtacagcagaagcagctagatatcgtggttgggcataa

SEQ ID NO: 73 - DNA, 1859 bp, Staphylococcus aureus
atgcctaaaaataaaattttaatttatttgctatcaactacgctcgtattacctactttagtttcacctaccgcttatgctgatacacctcaaaaagatactacagctaagacaacatctcatgattcaaaaaaatctaatgacgatgaaacttctaaggatactacaagtaaagatactgataaagcagacaacaataatacaagtaaccaagacaataacgacaaaaaatttaaaactatagacgacagcacttcagactctaacaatatcattgattttatttataagaatttaccacaaaccaatataaaccaattgctaaccaaaaataaatacgatgataattactcattaacaactttaatccaaaacttattcaatttaaattcggatatttctgattacgaacaacctcgtaatggcgaaaagtcaacaaatgattcgaataaaaacagtgacaatagcatcaaaaatgacactgatacgcaatcatctaaacaagataaagcagacaatcaaaaagcacctaaatcaaacaatacaaaaccaagtacatctaataagcaaccaaattcgccaaagccaacacaacctaatcaatcaaatagtcaaccagcaagtgacgataaagcaaatcaaaaatcttcatcgaaagataatcaatcaatgtcagattcggctttagactctattttggatcaatacagtgaagatgcaaagaaaacacaaaaagattatgcatctcaatctaaaaaagacaaaaatgaaaaatctaatacaaagaatccacagttaccaacacaagatgaattgaaacataaatctaaacctgctcaatcattcaataacgatgttaatcaaaaggatacacgtgcaacatcattattcgaaacagatcctagtatatctaacaatgatgatagcggacaatttaacgttgttgactcaaaagatacacgtcaatttgtcaaatcaattgctaaagatgcacatcgcattggtcaagataacgatatttatgcgtctgtcatgattgcccaagcaatcttagaatctgactcaggtcgtagtgctttagctaagtcaccaaaccataatttattcggtatcaaaggtgcttttgaagggaattctgttccttttaacacattagaagctgatggtaataaattgtatagtattaatgctggattccgaaaatatccaagcacgaaagaatcactaaaagattactctgaccttattaaaaatggtattgatggcaatcgaacaatttataaaccaacatggaaatcggaagccgattcttataaagatgcaacatcacacttatctaaaacatatgctacagatccaaactatgctaagaaattaaacagtattattaaacactatcaattaactcagtttgacgatgaacgcatgccagatttagataaatatgaacgttctatcaaggattatgatgattcatcagatgaattcaaacctttccgcgaggtatctgatagtatgccatatccacatggccaatgtacttggtacgtatataaccgtatgaaacaatttggtacatctatctcaggtgatttaggtgatgcacataattggaataatcgagctcaataccgtgattatcaagtaagtcatacaccaaaacgtcatgctgctgttgtatttgaggctggacaatttggtgcagatcaacattacggtcatgtagcatttgttgaaaaagttaacagtgatggttctatcgttatttcagaatccaatgttaaaggattaggtatcatttctcatagaactatcaatgcagctgccgctgaagaattatcatatattacaggtaaata

SEQ ID NO: 74 - DNA, 1092 bp, Staphylococcus aureus
ttattttaactttttagcgttatataaatcagctaaaggctttaattcatcataaaatgcttggaattcatctaaatccatttgttgacccgcatcactaagtgcaacagatggatctggatgcacctcagccataactccatcagcaccaactgctaatgctgctttcgcagttggtaacatgatatctttacgacctgtactatgcgtaacatctaccatgactggtaagtgtgtaccttgttttaaaattggtactgctgaaatatctaaagtgttacgtgtcgccttttcataagttcggattccacgttcacataaaataatgttttgattaccttgtgaagcaatgtattcagctgcataaacaaactcttcgattgtagcagataaaccacgttttaatagaataggctttttcgtacggccagcttcttttaataactcgaagttttgcatattacgtgcaccaatttggaatacgtctaaatactcatcagccacttcaaaatcatttggatttacgatttcgctgacaacatttaaatcatatttatctttaatctgtttaagtattttaagtccttcaacacctaggccttggaaatcatatggtgatgtacgtggtttaaatgcaccgccacgaataaatttttcacctttagcatgtaagtttttagcaacagcttcaacttgttcaaatgattcaactgaacatggcccaaatacaaatgatttattgccgtctccaataatgcccccattatcaaatgttacaatcgtatcttcaggtttcaacttacgtgatacgtataagtgtttttcattttcagatttttgtaaatctgtagaggctttaaaaatttctttaaataattgcttaattgtattatcgttaaatggtcctttgttgctatcaattaagtcgttaagcatttctttttcgcgttgtggatcatagatacgtgtaccttgttttaatttttcctccccaattttttgtgctagttcaccacgtttagataataaatctaaaatttgatgattcagtgagacaatctcacttctgtatgattctaatttattactcat

SEQ ID NO: 75 - DNA, 696 bp, Staphylococcus aureus
ttatgcagtaacccaatgtccagcgccaccagtgttatataattttactgctgcggcatcttgaacactttcaggagcatttgctggtgatacacctttatatttagcaggtgctactgaatcccaagttgattgtaagaattgatacttaccagctgcacctgatgttggatttacagcatgaatattgccacctgattcacgttgagcaatttgttttagatgagcattcacatttactgatgaaccttctgatgcttttgattcagttggtgttgcagtaacttgtgaattgtttgatgttgatgcttgtggttgttgagtttgagcattttgtggtgcttcaacttcttgtgattgtacttgattagcttgaacagctgatggttcaacattattagttgcaggtgcttgtgcactcatgtttgctccattagcacctgttgcatggtaattccaagcaaagtgtgtaccatctgattcaaagtgataagtaaacccttcatagtcaaatgtataattataagccccagcttcaattggtttttgatttaatgtttgatcatttgattgcgccatttgcgctaaagatgctttatttaagtccgcttcacttgcatgggcttcgtgacctgcatttcctgctacgattcctaaacctactgctaatgatgatgcgagtaatgttttcttcat

SEQ ID NO: 76 - DNA, 948 bp, Staphylococcus aureus
atggacatagaattaacaaaaaaagatggtactgtaatcaaattaagtgaatacgggtttatcgttaacgatatagtaattgatagtatgcaaatcaacacaaagtatcaagataaagaaaatatgaacggtcgtatattaatggggagcaattatatcagtagagatatagttgttccttgtttttgtgtggtaaaaaatcgttcagacattgcttatatgcgagatatgttgtattcgttaacgacagacatagaacctatgtatttgcgagaaataagaagaaaagaagagttgaattacaggtttactcaaccaatttctgatgattacgtgaaattagataaaaacaacttcccggattatgaatattcaagacacgatcaacaaaattatgtaaatggtaaacagtataaagttatttttaacggagttataaaccctaaacaaaaaggtaataaagtttcttttgaactaaaattcgaaactacagaattaccatacggtgaaagtattggaacaagcctagagttagaagaaaacaaaaaggttggattgtggtcgtttgattttaatattgattggcatgcaggcggagacaaaagaaagtatacatttgaaaatttgagcaaaggtacagtttactatcatggtagtgctcctaacgaccaattcaacatgtataaaaagataacaattattttaggcgaagatacagaatcgtttgtatggaatttaacgcatgctgaaataatgaaaatcgaagggatcaaactaaaaactggagacagaattgtttatgatagcttccgagtttataaaaacggtgttgaaataagtaccgaaacgaatatagcccaaccaaaatttaaatacggagctaataaatttgagtttaatcaaacggtacaaaaagttcagtttgatttgaaattttattataagtag

SEQ ID NO: 77 - DNA, 723 bp, Staphylococcus aureus
atggctcaaatttctaaatataaacgtgtagttttgaaactaagtggtgaagcgttagctggagaaaaaggatttggcataaatccagtaattattaaaagtgttgctgagcaagtggctgaagttgctaaaatggactgtgaaatcgcagtaatcgttggtggcggaaacatttggagaggtaaaacaggtagtgacttaggtatggaccgtggaactgctgattacatgggtatgcttgcaactgtaatgaatgccttagcattacaagatagtttagaacaattggattgtgatacacgagtattaacatctattgaaatgaagcaagtggctgaaccttatattcgtcgtcgtgcaattagacacttagaaaagaaacgcgtagttatttttgctgcaggtattggaaacccatacttctctacagatactacagcggcattacgtgctgcagaagttgaagcagatgttattttaatgggcaaaaataatgtagatggtgtatattctgcagatcctaaagtaaacaaagatgcggtaaaatatgaacatttaacgcatattcaaatgcttcaagaaggtttacaagtaatggattcaacagcatcctcattctgtatggataataacattccgttaactgttttctctattatggaagaagggaatattaaacgtgctgttatgggtgaaaagataggtacgttaattacaaaataa

SEQ ID NO: 78 - DNA, 774 bp, Staphylococcus aureus
atgagcttattatctaaaacgagagagttaaacacgttacttcaaaaacacaaaggtattgcggttgattttaaagatgtagcacaaacgattagtagcgtaactgtaacaaatgtatttattgtatcgcgtcgaggtaaaattttagggtcgagtctaaatgaattattaaaaagtcaaagaattattcaaatgttggaagaaagacatattccaagtgaatatacagaacgattaatggaagttaaacaaacagaatcaaatattgatatcgacaatgtattaacagtattcccacctgaaaacagagaattattcatagatagtcgtacaactatcttcccaattttaggtggaggagaaagattaggtacattagtacttggtcgagtacatgatgattttaatgaaaatgatttggtactaggtgaatatgctgctacagttattggtatggaaatcttacgtgagaagcatagtgaagtagaaaaagaagcgcgcgataaagctgctattacaatggcaattaattcattatcttattctgaaaaagaagcgattgaacatatctttgaagaacttggcggtacggaaggcctattaatcgcatcaaaagttgcagatagagttggtattactagatctgtaattgtaaatgcactacgtaaattagaaagtgctggtgtaattgaatcacgttctttaggaatgaaaggtactttcattaaagttaaaaaagaaaaattcttagatgaattagaaaaaagtaaataa

SEQ ID NO: 79 - DNA, 720 bp, Staphylococcus aureus
tcaactttcgtccttataagatatttctacatctaaatggaatttttcagtttcgacagttttgttgtcattatatatttttaagaaactttcagcttgtcctgttccagtatcaaataagtcataagaaaatgatgaaccatcattcaaatgaaataatatttttcctgcattaaatccagaactaaattttgacttattaccataatttcttcctttatttgtatcattaaagccataaatattatattcttcttgtagatatcttcttaatttagtatcaatttcttgagctgttactaactttttattagttgatactttgtcagtagatatagtttgatgttctccattaacccaaagattaataggaatacgtctagatttctctaaataatcacctgctaatgtaactccaccgtatatatatttgtttttcaaacaatatccactataacttagaccatatacatctacagcatgatttttgaatttctttacatcatcaacgttcttaaattcagctgttactgtcgaattatccatagaaaaaactaattgatgcgaaagatgattattttcaggattaatactttggtggtcttcaattggatagctaccataatagttcctaagattcaaaactccgacatcagcggttgcgatatgattttgcgaatagcaaaacaataaaacaacaatgataagtattcttttcat

SEQ ID NO: 80 - DNA, 798 bp, Staphylococcus aureus
atgaatcgtttgcatggacaacaagttaaaattggttacggggataacacgattataaataaattagatgttgaaataccagatggcaaagtgacgtcaatcattggtcctaacggctgcgggaaatctactttgctaaaggcattgtcacgtttattggcagttaaagaaggcgaagtatttttagatggtgaaaatattcatacacaatctacgaaagagattgcaaaaaaaatagccattttacctcaatcacctgaagtagcagatggcttaactgttggggaattagtttcatatggtcgttttccacatcaaaaaggatttggtagattaactgctgaggataagaaagaaattgattgggcaatggaagttacaggaactgatacattccgacaccgttcaatcaatgatttaagtggtggtcaaagacaacgtgtttggattgcaatggcattagcacaaagaactgatattatctttttagacgaaccaacaacatatttagatatctgtcatcaattagaaatactagaattagttcagaagctaaatcaggaacaaggttgtacaattgtcatggttcttcatgatatcaaccaagcgattcgtttctcagatcatcttattgcgatgaaagaaggggatatcatcgctacaggttcaacagaagacgtattaacacaggaaatattagaaaaagtttttaatattgatgttgttttaagtaaagatcctaaaactggaaaacctttactggtaacttatgacttatgtcgcagagcttattcttaa

SEQ ID NO: 81 - DNA, 957 bp, Staphylococcus aureus
atgaactatcaagttcttttatattataaatatacgacgattgatgaccctgaacagtttgctcaggatcacttagcgttttgcaaagcacaccatttaaaaggtagaattcttgtttctacagaaggtattaacggcacattatctggtacaaaagaagaaaccgaacaatatatggcacatatgcatgccgatgaacgattcaaagatatggtgtttaaaattgatgaagctgaaggacatgcttttaagaaaatgcatgtacgtcctcgaaaagaaatcgttgctttagatttagaagatgacgtcgatccaagacacacaactggccaatatttatcacctgtagaatttagaaaagctcttgaagatgatgacacagtcattattgatgcacgtaatgattatgaatttgatttaggtcatttccgaggtgcaattcgcccagacatcacacgctttagagatttacctgactggattaaagagaataaagcgttatttacagataaaaaagtggttacgtactgtacaggtggcattcgatgtgaaaaattttctggatggcttttaaaagaaggtttcgaagatgtagcgcagcttcacggcggtatagctacatatggtaaagaccctgaaacaaaaggtcaatattgggacggtaaaatgtatgtatttgatgatcgtatcagtgttgatatcaaccaagttgaaaaaacaattattggtaaggattggtttgatggcaaaccatgtgaacgttatattaattgcgctaacccagaatgtaataaacaaatattagtttctgaagaaaacgaaactaaatatttaggtgcatgctcttatgaatgtgctaaacatgagcgtaatcgttatgttcaagcaaataatattagtgataatgagtggcaacaacgtttaacaaactttgatgatttacatcaacatgcttag

SEQ ID NO: 82 - DNA, 654 bp, Staphylococcus aureus
atggcttcattaaagtcaatcatccgtcaaggtaaacaaacacgttcagatcttaaacaattaagaaaatctggtaaagtaccagcagtagtatacggttacggtactaaaaacgtgtcagttaaagttgatgaagtagaattcatcaaagttatccgtgaagtaggtcgtaacggtgttatcgaattaggcgttggttctaaaactatcaaagttatggttgcagactaccaattcgatccacttaaaaaccaaattactcacattgacttcttagcaatcaatatgagtgaagaacgtactgttgaagtaccagttcaattagttggtgaagcagtaggcgctaaagaaggcggcgtagttgaacaaccattattcaacttagaagtaactgctactccagacaatattccagaagcaatcgaagtagacattactgaattaaacattaacgacagtttaactgttgctgatgttaaagtaactggcgacttcaaaatcgaaaacgattcagctgaatcagttgtaacagtagttgctccaactgaagaaccaactgaagaagaaatcgaagctatggaaggcgaacaacaaactgaagaaccagaagttgttggcgaaagcaaagaagacgaagaaaaaactgaagagtaa

SEQ ID NO: 83 - DNA, 762 bp, Staphylococcus aureus
atgagaacaccaattatagctggtaactggaaaatgaacaaaacagtacaagaagcaaaagacttcgtcaatgcattaccaacattaccagattcaaaagaagtagaatcagtaatttgtgcaccagcaattcaattagatgcattaactactgcagttaaagaaggaaaagcacaaggtttagaaatcggtgctcaaaatacgtatttcgaagataatggtgcgttcacaggtgaaacgtctccagttgcattagcagatttaggcgttaaatacgttgttatcggtcattctgaacgtcgtgaattattccacgaaacagatgaagaaattaacaaaaaagcgcacgctattttcaaacatggaatgactccaattatttgtgttggtgaaacagacgaagagcgtgaaagtggtaaagctaacgatgttgtaggtgagcaagttaagaaagctgttgcaggtttatctgaagatcaacttaaatcagttgtaattgcttatgagccaatctgggcaatcggaactggtaaatcatcaacatctgaagatgcaaatgaaatgtgtgcatttgtacgtcaaactattgctgacttatcaagcaaagaagtatcagaagcaactcgtattcaatatggtggtagtgttaaacctaacaacattaaagaatacatggcacaaactgatattgatggggcattagtaggtggcgcatcacttaaagttgaagatttcgtacaattgttagaaggtgcaaaataa

SEQ ID NO: 84 - DNA, 1062 bp, Staphylococcus aureus
ttatgttaaaactataaggtcttttgtgcatttagtaaagacttgacaaccattttctgtaattaaaatatcatcttctattcttataccgcccaaaccttctatataaacaccaggttctactgtaacacagttgttaacttgaagtttatcttgtatcgtacgagccagcattggcccttcatggatttctaaaccaataccatgtcctagtgaatgtccaaattcttttccatacccttttgactctaaatagtttcttgaaatggcatcagcttctgcaccagtcatgccaggtctaatctcattaattgctttcatttgagattcaagtactatttgatatatttctttcagtttaggatctggttctccaatagcaaatgttctagtaatatctgaacaatagccgttataatacgcgccaaaatctaatgtaatcatgtcgcctttttcaataattttatcacttgcaacaccatgtggtaatgcacctctatgaccagatgctacaatcgtatcaaatgatggtccatctgctcctaattctagcattttactttctaatattgcctttaattctttttcagtcatacctgcttttacaacagttaaaatatattcatatgtttcatcaacaatattagctgctttttgaattaaagcaatttcgtcaacatctttgacgtctctaattttatctacagtattagaaatgcttattaatgatatacggcttttatttaattcaaggtatgtatcataacttacatgatgcccctcaaaacctatattttcaaaattttcttggtgtagcaattctttaatctcaccaataatagtagatttacgattaataatttcataatttggcgcctgcttagttgcttgatcaatatatctaaagtctgttatcaaatattgtttatctttagatatgataagtgctccactggtaccagtaaaacctgataaatatcttctattgtaatccgaaagaatgataatcgcatctaaatgtttttgttctaaaatacgatgcacttgtgttattctgctcat